(19) United States
(12) Patent Application Publication
(10) Pub. No.: US 2024/0052079 A1
(43) Pub. Date: Feb. 15, 2024

(54) ANTIBACTERIAL ESTER-FREE MONOMERS FOR DENTAL ADHESIVES

(71) Applicant: OREGON HEALTH & SCIENCE UNIVERSITY, Portland, OR (US)

(72) Inventors: Carmem Pfeifer, Beaverton, OR (US); Wilbes Mbiya, Portland, OR (US); Ana Paula Piovezan Fugolin, Portland, OR (US); Jack Ferracane, Beaverton, OR (US); Justin Merritt, Portland, OR (US); Matthew Logan, Portland, OR (US)

(21) Appl. No.: 18/269,150

(22) PCT Filed: Feb. 22, 2022
(86) PCT No.: PCT/US2022/017354

§ 371 Date: Jun. 22, 2023

Related U.S. Application Data
(60) Provisional application No. 63/152,249, filed on Feb. 22, 2021.

(51) Int. Cl. C08F 222/38; C08F 2/50

(52) U.S. Cl. CPC C08F 222/385; C08F 2/50; A61K 6/30

(57) ABSTRACT

Provided herein are novel compounds of Formula (I):

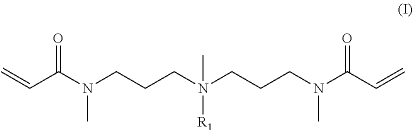

wherein R₁ is selected from the group of C₁-C₂₀ alkyl, —CH₂—CH₂—CH₂—NH—C(=O)—C=C, and —CH₂—CH₂—CH₂—N(CH₃)—C (=O)—C=C; along with compositions comprising them and uses, including as dental adhesive compositions.

### STATEMENT OF GOVERNMENT SUPPORT

This invention was made with government support under 1U01-DE023756, 1R01-DE026113, K02-DE025280; R01-DE028757; and R35-DE029083 awarded by NIH-NIDCR. The government has certain rights in the invention.

### FIELD OF THE INVENTION

The present invention concerns quaternary ammonium (meth)acrylamides as antibacterial monomers for dental applications.

### BACKGROUND OF THE INVENTION

Dental adhesives are widely used in restorative dentistry to provide micromechanical and chemical retention between restorative resin-based composites and the dental substrate. Despite the significant advancements achieved for dental adhesive systems, the degradation of the hybrid layer remains a challenge for the longevity of the bond at the dentin-adhesive interface. This degradation process is complex and multifactorial, and involves both the hybrid layer constituents (mainly the collagen fibers) and the adhesive polymer [1].

Though not without controversy, the collagen degradation has been hypothesized to be due to proteolytic activity of host-derived matrix metalloproteinases (MMPs) [1-3] and cysteine cathepsins (CPs), which together have a synergistic collagenolytic/gelatinolytic activity [1, 4, 5]. The adhesive polymer layer may be degraded by water percolation, in addition to the action of proteolytic enzymes capable of hydrolyzing the ester bonds in the methacrylate-based materials [6-8]. Ester groups have shown accelerated degradation in the presence of esterases such as cholesterol esterase (CE) and pseudocholinesterase (PCE) [9]. In addition, the presence of residual water compromises the polymerization of adhesive monomers, leading to phase separation and loss of adhesive integrity early in the bonding process [10, 11]. Additionally, the copolymerization of HEMA with a hydrophobic difunctional methacrylate (such as BisGMA and UDMA) forms a loosely cross-linked network, with hydrophilic HEMA-rich domains that are easily plasticized by swelling with water. Also, due to its low molecular weight and the tendency to partition in water, HEMA is easily extracted from the polymer network, with consequent biological repercussions, such as suppression of expression of certain genes associated with protection against excessive cell proliferation, apoptosis and cell mutation [13]. Other than HEMA, the dimethacrylates, commonly used as base monomers can also pose concern in terms of cytotoxicity. Once hydrolyzed, BisGMA may result in the formation of several by-products, including bis-HPPP—a toxic dialcohol, and methacrylic acid [8, 14]. However unlikely, according to several recent studies [15, 16], the potential release of estrogenic BPA (bisphenol A) from the BisGMA degradation is a persistent concern.

Considering the shortcomings of current materials, the use of methacrylamides, in particular multifunctional crosslinkable amides, have been proposed as components of adhesives [17]. For example, one diacrylamide (DEBAAP—N,N-Diethyl-1,3-bis(acrylamido)propane) was proposed as a hydrolytically—stable monomer as a replacement for methacrylates in self-etching adhesives and composites [18]. When used in the formulation of an adhesive, this monomer produced shear bond strength approximately 20% higher than the pure methacrylate control. When used in the formulation of a composite, the material had comparable flexural strength/modulus, with the advantage of a 35-fold decrease in cytotoxicity compared with TEGDMA-containing formulations. Another recent study on the use of acrylamides and methacrylamides in etch-and-rinse dental adhesives demonstrated increased stability of the dentin bond strength, despite experiencing a decrease in mechanical properties after storage in water [17]. In this same study, the monomer structure was systematically varied to highlight the effect of steric hindrance and electronic factors, and the results showed that tertiary methacrylamides, although more hydrolytically and enzymatically stable than the acrylam ides, presented markedly lower reactivity [17]. Taken together, these results demonstrate that, in spite of the greater susceptibility to water sorption, the use of (meth)acrylamides as ester-free monomers to totally or partially replace methacrylates is a promising strategy to tune polymer properties, and ultimately produce dental adhesive systems with longer clinical service.

Using analog monomers of similar functionality is paramount to understand the influence of the methacrylamide on the function of these adhesives. This was indeed the topic of a previous study, where systematically modified mono-acrylamides and methacrylamides are compared with HEMA and HEA (2-hydroxyethyl acrylate). The goal of the present study was to move the field one step further, by adding multi-functional, crosslinkable monomers to the composition, and compare those to the gold standard in dental adhesive formulations. Tert-amine core-bearing multi-functional acrylamides were designed and synthesized, and usee in BisGMA-free dental adhesive formulations. Kinetics of polymerization, water stability, mechanical properties, dentin bond strength, cytotoxicity and biofilm formation were evaluated. The tested hypothesis was that the newly synthesized multi-functional acrylamides would provide greater hydrolytic stability, translating into enhanced bond stability while maintaining other properties comparable to the methacrylate control.

### SUMMARY OF THE INVENTION

An embodiment herein provides compounds of Formula (I):

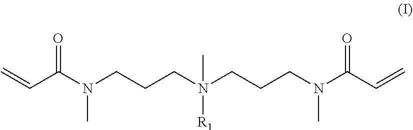

wherein R₁ is selected from the group of C₁-C₂₀ alkyl, —CH₂—CH₂—CH₂—NH—C(=O)—C=C, and —CH₂—CH₂—CH₂—N(CH₃)—C (=O)—C=C.

Additional embodiments also provide compositions comprising and formed using a compound of Formula (I).

### BRIEF DESCRIPTION OF THE DRAWINGS

FIG. 1 provides structures of tested monomers HEMA, DEBAAP, BMAAPMA, TMAAEA, and BAADA.

FIG. 2A provides a graph of comparative rates of polymerization as a function of degree of conversion for tested formulations.

FIG. 2B provides a bar graph of water sorption (WS) for tested compositions.

FIG. 2C provides a bar graph of Solubility (SL) results for tested compositions.

FIG. 3 shows a representative example of the NMR spectra and integration regions used to calculate monomer degradation.

FIG. 4 presents a graph of degradation kinetics over one month as well as the final degradation percentage for each tested monomer in acidic aqueous conditions (pH=1).

FIG. 5A presents a bar graph representing the biocompatibility of tested compounds evaluated with undifferentiated pulp cells (OD-21)

FIG. 5B presents a bar graph representing the biocompatibility of tested compounds evaluated with human dental pulp stem cells (h-DPSC).

FIG. 6A presents a bar graph of comparative luciferase assay results for tested compounds.

FIG. 6B graphs comparative biofilm adhesion as measured by an impingement test.

FIG. 7 presents a table of mean and standard deviation of degree of conversion (DC in %), maximum rate of polymerization (Rpmax in %.s−1), DC at Rpmax (in %), flexural strength (FS, MPa), and elastic modulus (E, GPa) for tested compositions.

FIG. 8 presents a table of mean and standard deviation of microtensile bond strength tests (MPa) for adhesive formulations after storage in distilled water at 37° C. for 24 hours, 3 weeks and 6 months.

FIG. 9 provides box plot graphs of water sorption and solubility (μg/mm³) for tested formulations.

FIG. 10A provides structure of quaternized monomers BMAAPMA-C4, BMAAPMA-C6, and BMAAPMA-C14.

### DETAILED DESCRIPTION OF THE INVENTION

An embodiment herein provides compounds of Formula (I):

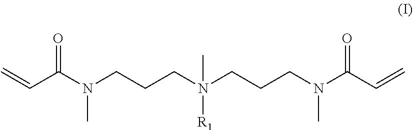

wherein R₁ is selected from the group of C₁-C₁₀ alkyl, —CH₂—CH₂—CH₂—NH—C(=O)—C=C, and —CH₂—CH₂—CH₂—N(CH₃)—C (=O)—C=C.

A further embodiment provides compounds of Formula (I), wherein R₁ is selected from the group of C₁₀-C₂₀ alkyl.

Another embodiment provides compounds of Formula (I), wherein R₁ is selected from the group of C₁₀C₁₆ alkyl.

Another embodiment provides compounds of Formula (I), wherein R₁ is selected from the group of C₁₂-C₁₆ alkyl.

An additional embodiment provides a compound of Formula (I), wherein R₁ is —CH₂—CH₂—CH₂—NH—C(=O)—C=C, which may also be represented by the formula below, wherein the wavy line “ [symbol] ” represents the bond through which the designated Ri moiety is bound to the central quaternary nitrogen atom:

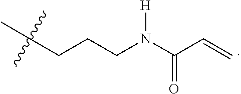 [SMILES: *CCCNC(=O)C=C]

An additional embodiment provides a compound of Formula (I), wherein R₁ is —CH₂—CH₂—CH₂—NH—C(=O)—C=C, which may also be represented by the formula below, wherein the wavy line “ [symbol] ” represents the bond through which the designated R₁ moiety is bound to the central quaternary nitrogen atom:

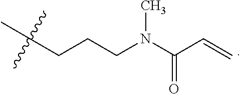 [SMILES: *CCCN(C)C(=O)C=C]

Non-limiting examples of compounds of Formula (I) include:

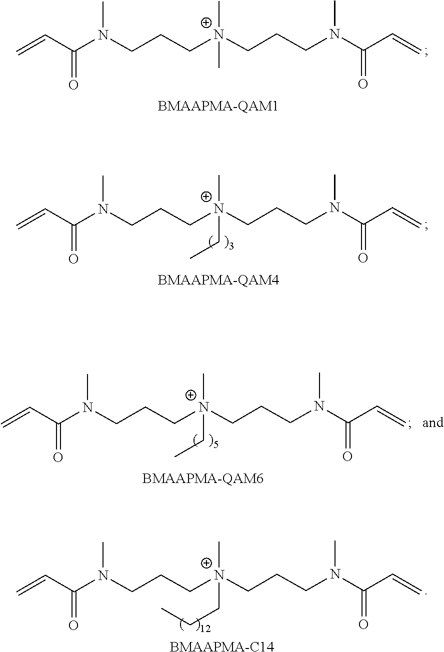

Other embodiments, provide a composition comprising a monomer compound of Formula (I) and one or more co-monomers selected from the group of bisphenol A diglycidyl ether dimethacrylate (BisGMA), triethylene glycol dimethacrylate (TEGDMA), urethane dimethacrylate (UDMA), ethylene glycol dimethylacrylate (EGDMA), ethane-1,2-diyl bis(2-methylacrylate) (PEGDMA) ; ethoxylated bisphenol A dimethacrylate (EBPADMA), ethylene glycoldi(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, 1,6-hexanediol dimethacrylate (HEDMA), 1,6-hexamethylene glycol dimethacrylate (HGDMA), divinyl benzene and derivatives thereof.

In some embodiments, the monomer compound of Formula (I), or a salt thereof, comprises from about 35% to about 45%, by weight, of the composition. In some embodiments, the co-monomer or co-monomers selected from this group comprises from about 55% to 65% of the composition, by weight. In some embodiments, the monomer compound of Formula (I), or a salt thereof, comprises from about 37% to about 43%, by weight, of the composition. In some embodiments, the co-monomer or co-monomers selected from this group comprises from about 57% to 63% of the composition, by weight.

Still other embodiments provide such compositions comprising at least one monomer and one co-monomer, as described herein, and further comprising a polymerization initiator, such as one selected from the group of camphorquinone (CQ); trimethylbenzoyl-diphenyl-phosphine oxide (TPO); Ethyl-4-dimethylamino benzoate (EDMAB); 2,2-Dimethoxy-2-phenylacetophenone (DMPA); Bisacylphosphine oxide (BAPO); 1-Phenyl-1,2-propanedione (PPD); phosphine oxide compounds, including naphthacene (APO), 9-anthracene (APO), and bisacyiphosphine oxide (BAPO); 1-phenyl-1,2.-propanedione (PPD); thioxanthone (TX) and its derivatives; a dibenzoyl germanium derivative, benzoyltrimethylgermane (BTG), dibenzovidiethyigermane; hexaarylbiimidazole derivatives; a silane based derivative; (diethyigermanediyi)bis((4-methoxyphenyl)methanone); benzenesulfinic acid sodium salt (BS); a diaryliodonium salt, diphenyliodoniurn chloride or iodonium salt [diphenyliodonium hexafluorophosphate (DPIHP ar DPI-PF6))], bromide, iodide or hexafluorophosphate; benzoyl peroxide (BPO), and ethyl 4-N,N-dimethaminobenzoate. In further embodiments, the polymerization initiator is a combination of initiators, such as those selected from the group of camphoquinone/ethyl-4-(dimethylamino)benzoate (EDMAB), camphorquinone/2-(dimethylamino)ethyl methacrylate (DMAEMA)), DMPA/DPI-PF6, CQ/PPD, CQ/DMAEMA, CQ/EDMAB, CQ/DMAEMA/PDIHP, or CQ/EDMAB/DPIHP. In some embodiments, the polymerization initiator one or both of the group DMPA and DPI-PF. In some embodiments, the polymerization initiator comprises from about 0.05% to about 0.6% of the composition, by weight.

In some embodiments, the composition also comprises a chemical inhibitor (also referred to as a stabilizer or free radical scavengers), such as one selected from the group of butylated hydroxytoluene (BHT), hydroquinone, 2,5-di-tert-butyl hydroquinone, monomethyl ether hydroquinone (MEHQ), and 2,5-di-tertiary butyl-4-methylphenol, 3,5-di-tert-butyl-4-hydroxyanisole (2,6-di-tert-butyl-4-ethoxyphenol), 2,6-di-tert-butyl-4-(dimethylamino)methylphenol or 2-(2′-hydroxy-5′-methylphenyl)-2H -benzotriazole, 2-(2′-hydroxy-5′-t-octylphenyl)-2H-benzotriazole, 2-(2′-hydroxy-4′,6′-di-tert-pentylphenyl) -2H-benzotriazole, 2-hydroxy-4-n-octoxybenzophenone, 2-(2′-hydroxy-5′-methacryloxy-ethylphenyl) -2H-benzotriazole, phenothiazine, and HALS (hindered amine light stabilizers).

The compositions may also comprise an ultraviolet light (UV) absorber, such as 2-hydroxy-4-methoxybenzophenone (UV-9), 2-(2-Hydroxy-5-octylphenyl)-benzotriazole (UV-5411), salicylic add phenyl ester, 3-(2′-hydroxy-5′-methylphenyl)benzotriazole, and 2-(2′-hydroxy-5′-methylphenyl)-benzotriazole, The UV absorber may be present in the composition at from about 0.001% to about 0.5%, by weight.

In some embodiments the chemical inhibitor is incorporated into the composition at a concentration of from about 0.01% to about 0.5%, by weight. In other embodiments, the chemical inhibitor is present in the composition at from about 0.05% to about 0.3%, by weight. In still other embodiments, the chemical inhibitor is present in the composition at from about 0.05% to about 0.2%, by weight. In additional embodiments, the chemical inhibitor is present in the composition at from about 0.05% to about 0.15%, by weight.

it is understood that the compositions herein may include further elements, such as a fluorescent agent, a fluoride releasing agent, a radiopaque agent, a flavoring agent, and an antimicrobial agent.

### Materials and Methods

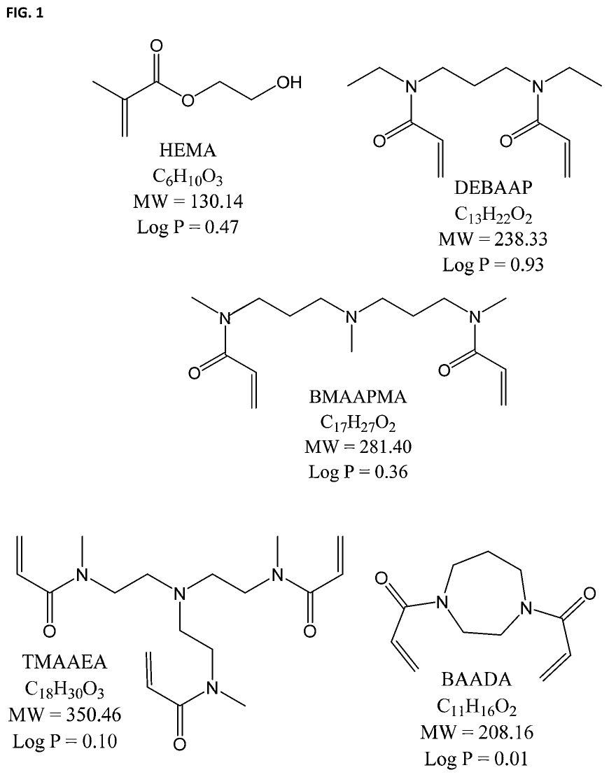

Experimental adhesives were formulated with 60 wt % UDMA and 40 wt % of one of the monomers shown in FIG. 1. The rationale for the selection of these specific monomers was a combination of the greater reactivity of acrylamides in comparison with methacrylamides [17], and the potential for increased crosslinking with the multi-functional monomers. Therefore, three novel tert-amine multi-functional acrylamides, N,N-bis[(3-methylaminoacryl)propyl]methylamine (BMAAPMA), tris[(2-methylaminoacryl)ethyl]amine (TMAAEA) and N,N′-bis(acrylamido) 1,4-diazepane (BAADA) were synthesized de novo, as described below. N,N-Diethyl-1,3-bis(acrylamido)propane (DEBAAP) was synthesized as described previously [18] to serve as the experimental acrylamide control. HEMA-containing materials were tested as methacrylate controls. The photoinitiator system was composed by 0.2 wt % 2,2-dimethoxy-2-phenylacetophenone (DMPA) and 0.4 wt % diphenyl iodonium hexafluorophosphate (DPI-PF6). This initiator system was based in a previous study [19], which demonstrated its better efficiency compared with a conventional camphorquinone/amine system. Butylated hydroxytoluene (BHT, 0.1 wt %) was added to each formulation as an inhibitor to improve shelf life.

### Monomers Synthesis

Reagents for the synthesis reactions were used as received (obtained from Sigma Aldrich, Milwaukee, WI, unless otherwise noted), except Acetonitrile (ACS grade), which was dried over 3 Å molecular sieves for at least 24 h prior to use.

Reactions were carried out in anhydrous MeCN and on a blanket of N₂. Acryloyl chloride was freshly distilled before use. ¹H and ¹³C NMR spectra were acquired with a Bruker AMX (400 and 100 MHz, respectively). ¹H NMR chemical shifts are reported as values in ppm relative to CHCl₃ (7.26) in CDCl₃, H₂O (4.79) in D₂O and Me₄Si (TMS) was used as internal standard. Chemical shifts are reported as δ values in parts per million (ppm) and coupling constants (J) are reported in hertz (Hz).

N,N-Bis[(3-methylaminoacryl)propyl]methylamine (BMAAPMA): Acryloyl chloride (90.0 mmol, 7.35 mL) and MEHQ (2.0 mg) were dissolved in 150 mL of anhydrous MeCN. The flask was placed in an ice bath between −5° C. and −10° C. before dropwise addition of N,N-bis[3-(methylamino)propyl]methylamine (90.0 mmol, 15.6 g) dissolved in 45 mL of MeCN. After 48 hours, the excess amine formed an insoluble hydrochloride salt and was filtered. The filtrates were concentrated in vacuo and the remaining oil was re-dissolved in DCM and washed in sequence with 0.1 M HCI aqueous solution, saturated NaHCO₃ aqueous solution and water. The remaining organic layer was dried over MgSO₄ and concentrated in vacuo, yielding 6.99 g of a slightly yellow oil (24.8 mmol, 55.1% yield). ¹H NMR (400 MHz, Deuterium Oxide) δ 6.81- 6.60 (m, 2H), 6.21-6.06 (m, 2H), 5.86-5.70 (m, 2H), 3.56-3.34 (m, 4H), 3.11 (s, 3H), 2.97 (s, 3H), 2.48- 2.30 (m, 3H), 2.28-2.12 (m, 3H), 1.88-1.67 (m, 4H). ¹³C NMR (101 MHz, Deuterium Oxide) δ 169.01, 168.77, 128.36, 128.28, 127.70, 127.40, 53.56, 53.50, 53.17, 53.10, 48.39, 46.28, 46.24, 40.90, 40.82, 35.59, 33.91, 24.74, 24.72, 23.40, 23.34. ITMS m/z Calcd (C₁₅H₂₈N₃O₂⁺): 282.22; experimental 282.21 (Figure S1).

Tris[(2-methylaminoacryl)ethyl]amine (TMAAEA): Acryloyl chloride (40.0 mmol, 3.24 mL) and MEHQ (1.0 mg) were dissolved in 75 mL of anhydrous MeCN. The flask was placed in an ice bath between −5° C. and −10° C. before dropwise addition of tris[2-(methylamino)ethyl]amine (26.5 mmol, 5.00 g) dissolved in 20 mL of MeCN. After 48 hours, the excess amine formed an insoluble hydrochloride salt and was filtered. The filtrates were concentrated in vacuo and the remaining oil was re-dissolved in DCM and washed in sequence with 0.1 M HCI aqueous solution, saturated NaHCO₃ aqueous solution and water. The remaining organic layer was dried over MgSO₄ and concentrated in vacuo, yielding 3.05 g of a colorless oil (8.70 mmol, 65.7% yield). ¹H NMR (400 MHz, Chloroform-d) δ 6.73-6.45 (m, 3H), 6.39-6.09 (m, 3H), 5.79-5.54 (m, 3H), 3.61-3.29 (m, 6H), 3.15-2.92 (m, 9H), 2.81-2.56 (m, 6H). ¹³C NMR (101 MHz, Deuterium Oxide) δ 169.15, 168.86, 128.55, 128.42, 127.62, 127.40, 52.05, 51.74, 51.33, 50.35, 50.17, 49.92, 48.28, 48.07, 47.88, 45.67, 45.56, 45.48, 36.14, 34.42. ITMS m/z Calcd (C₁₈H₃₁N₄O₃⁺): 351.24; experimental 351.23 (Figure S2).

N,N′-bis(acrylamido) 1,4-diazepane (BAADA): Acryloyl chloride (50.0 mmol, 4.04 mL) and MEHQ (1.0 mg) were dissolved in 75 mL of MeCN. The flask was placed in a water bath between −5° C. and −10° C. before the dropwise addition of 1,4-diazepane (50.0 mmol, 5.01 g) dissolved in 20 mL of MeCN was attached. After 48 hours, the excess amine formed an insoluble hydrochloride salt and was filtered. The filtrates were concentrated in vacuo resulting in 2.99 g of white crystalline solid (14.3 mmol, 57.3% yield). NMR (400 MHz, Deuterium Oxide) δ 6.82-6.61 (m, 2H), 6.28-6.05 (m, 2H), 5.91-5.71 (m, 2H), 3.88-3.71 (m, 4H), 3.72-3.56 (m, 4H), 1.97-1.73 (m, 2H). ¹³C NMR (101 MHz, Deuterium Oxide) δ 168.96, 168.86, 168.76, 168.73, 129.15, 129.10, 128.79, 128.79, 127.25, 127.17, 127.02, 126.92, 48.43, 48.06, 47.08, 46.96, 46.42, 46.03, 45.55, 45.47, 27.78, 26.11, 24.34. Multiplets in the ¹H spectra and duplicate peaks in the ¹³C spectra were observed suggesting multiple isomeric forms due to the ring structure of BAADA (Figure S3).

In addition, the diacrylamide BMAAPMA was also quaternized with side chains of 4, 6 and 14 carbons in an attempt to render the materials antimicrobial. The effect of side chain length in the antimicrobial potential has been demonstrated previously [20]. The synthesis procedure was the same as described above, with an additional quaternization step which can be found in supplementary information. ¹H spectra and duplicate peaks in the ¹³C spectra are shown in Figures S4-6.

In the instances where these monomers were tested as part of the composition, they were added at 10 wt % to the monomer matrix, with 60 wt % UDMA+30 wt % BMAAPMA. Control formulations (UDMA+HEMA and UDMA+BMAAPMA, 60:40, respectively), photoinitiator system, and photocuring conditions were the same as described throughout the Materials and Methods section.

### Polymerization Kinetics

Materials (10 mm in diameter and 0.8 mm in thickness, n=3) were sandwiched between two glass slides, then placed in the chamber of an Fourier Transform Infra-red spectrometer (Nicolet 6700, ThermoScientific, USA) and irradiated for 300 s with a mercury arc lamp (Acticure, EXFO Acticure 4000 UV Cure; Mississauga, Ontario, Canada) filtered to 320-500 nm and delivering 630 mW/cm² (irradiance reaching the surface of the sample from a distance of 1.5 cm, imposed by the IR set up). Spectra were collected in real-time during photoactivation, with 2 scans per spectrum at 4 cm⁻¹ resolution for 5 min. The degree of conversion was calculated based on the area of the methacrylate (6165 cm⁻¹) and acrylamide (6139 cm⁻¹) vinyl overtones, and the rate of polymerization was calculated as the first derivative of the conversion versus time curve [21].

### Water Sorption and Solubility

The same samples used in the polymerization kinetics tests were subjected to water sorption (WS) and solubility (SL) tests, according to ISO 4049 [22]. The initial “dry” mass (M1) was recorded before samples were immersed in 5 ml of Millipore water for one week. At the end of this time, they were carefully wiped off with absorbent paper, and weighed to record their mass after immersion (M2). Samples were then placed in a desiccator under vacuum until mass stabilization (M3). Water sorption (WS) and solubility (SL) were calculated according to Equations 1 and 2, respectively (V is the volume of the cylindrical specimen), reported in μg/mm³:

$$WS = 1000 x \frac{M 2 - M 3}{V} \qquad Equation ( 1 )$$

$$SL = 1000 x \frac{M 1 - M 3}{V} \qquad Equation ( 2 )$$

### Flexural Strength (FS) and Elastic Modulus (E)

For the flexure tests, twelve beam-shaped specimens (2.0×2.0×25.0 mm) per group were produced by filling silicone molds placed between two glass slides and photocuring for 120 s on each side with the mercury arc lamp at 630 mW/cm². Samples were tested in three-point bending after dry storage at room temperature for 24 hours and after 7 days water immersion (n=6). Crosshead speed of 0.5 mm/min until fracture was applied, according to ISO 4049.

### Dentin Microtensile Bond Strength

For bonding procedures, 40 vol % of ethanol was added to the resin formulations. Adper Single Bond 2 (3M ESPE) was tested as a methacrylate-based commercial control. According to the Safety Data Sheet [23], Single Bond is composed of BisGMA (10-20 wt %), HEMA (5-15 wt %), glycerol 1,2 dimethacrylate (5-10 wt %), UDMA (<5 wt %), ethyl alcohol (25-35 wt%), water (<5%), diphenyliodonium hexafluorophosphate (<0.5 wt %), methacrylate functional copolymer of polyacrylic and poly(itaconic) acids (5-10 wt %), and 5-nm-diameter spherical silica particles (10-20 wt %).

As substrate, caries-free extracted human third molars (n=6) were collected, cleaned and kept in 0.5% chloramine T. This study was cleared by the Oregon Health & Science University IRB (IRB00012056).

After enamel removal by cutting on saw, a flat dentin surface was wet polished with 600-grit silicon-carbide paper for 30 s to standardize the smear layer. The dentin surface was etched with 37% phosphoric acid (Scotchbond Universal Etchant, 3M ESPE) for 15 s, rinsed with water 15 s and carefully dried with cotton pellets in order to maintain the surface slightly wet. The first adhesive coat was vigorously applied for 20 s, the solvent evaporated by a gentle air stream for 10 s and a second adhesive coat was applied for 10 s and photocured for 60 s using the mercury arc lamp. This lamp was used for this layer to overlap the absorption spectrum of the initiator (DMPA−λₘₐₓ=365 nm). The overall irradiance was 630 mW/cm², and the irradiance for the peak at 365 nm was 330 mW/cm², as determined using a calibrated bench-top UV-Vis spectrometer (MARC Resin Calibrator, Blue Light analytics, Halifax, NS, Canada) [24]. Therefore, the effective radiant exposure delivered to the adhesive was approximately 20 J/cm². Adper Single Bond (3M ESPE) was applied and photocured according to the manufacturer's instructions. In summary, after phosphoric etching for 15 s, rinsing and drying, a first layer of the adhesive was actively applied for 15 s and the solvent evaporated. A second layer was applied and after solvent evaporation, it was photoactivated by a curing light (Demi™ Plus; Kayo Kerr) at 550 mW/cm² for 10 s (radiant exposure of 5.5 J/cm²). It is recognized that the radiant exposures were different for the materials manipulated in house and the commercial adhesive. This is an additional reason for the use of an “experimental control”, using similar initiator concentrations, and cured under identical conditions as the novel materials. We opted for keeping the commercial adhesive, cured under commercially recommended conditions as a benchmark. For either adhesive, resin blocks (Filtek Supreme, shade body A2) were built on the bonded surfaces in two-2 mm thick increments each photocured for 20 seconds (Demi™ Plus; Kayo Kerr) at 550 mW/cm².

The teeth were immersed in distilled water and kept at 37° C. for 24 h, and then sectioned perpendicular to the interface using a water-cooled diamond saw (Accutom-50; Struers) to obtain resin-dentin sticks with rectangular cross-sectional area of approximately 1.0 mm². The sticks were immersed in distilled water at 37° C. and tested after 24 hours, 3 weeks and 6 months.

The sticks were loaded in tension to failure in a universal testing machine (Criterion, MTS Systems Co., Eden Prairie, MN, USA) at a crosshead speed of 0.5 mm/min. The bonded surface area was calculated after measuring each stick with a digital caliper (Mitutoyo, Tokyo, Japan). Each stick was attached to the grips of a microtensile jig ensuring uniaxial alignment of the stick with the loading device (Odeme Equipamentos, Sao Paulo, SP, Brazil) with a cyanoacrylate adhesive (Zap It Super Glue; Henkel/Loctite, Westlake, Ohio, USA). The results were recorded in MPa and sticks for each tooth were averaged to provide one value per tooth.

### Hydrolytic Degradation

Neat monomers were subject to the hydrolytic degradation kinetics assay. An aqueous solution (pH=1) was prepared using HPLC grade water and adjusted using 1.0 M HCI. A 50 mM solution of each monomer (n=3) was prepared using 1.0 mL of the acidic aqueous solution. A capillary tube was filled with a 50 mM solution of tetramethylammonium bromide dissolved in D₂O and flame sealed. The capillary tube was placed at the bottom on the NMR tube to allow the “locking on” of the instrument and to act as an internal standard. ¹H NMR spectra were obtained using a water suppression by excitation sculpting experiment. After the initial reading, the NMR tubes were flame sealed and incubated at 37° C. During the first week and after 12, 19, and 30 days, the samples were removed from incubation to obtain water suppressed ¹H NMR spectra. To determine the amount of monomer degradation, spectra were first aligned using the ITSD singlet peak and then an integration region unique to a monomer vinyl proton and an integration region unique to a degradation product (methacrylic or acrylic acid) vinyl proton were used to calculate the ratio of monomer to degradation product. An integration region where a vinyl peak is found in both TMAAEA and acrylic acid was used to normalize the integration values. Additional integration regions on either side of the vinyl region were used to determine the average amount of noise in each spectra. Degradation product values with a signal to noise ratio of less than 10 were considered to show no measurable amount of degradation.

### Cytotoxicity

For the cytotoxicity assay, two cell types were tested—odontoblast-like cell line (OD-21) and human dental pulp stem cells (hDPSC) (cat #PT5025, Lonza). OD-21 cells were cultured in high glucose Dulbecco's Modified Eagle's medium without phenol red (DMEM, cat #21063-029, Thermo Fisher Scientific, Grand Island, NY, USA) and hDPSCs were cultured in alpha Minimal Eagle's medium (α-MEM) (Gibco, Waltham, MA, USA), both supplemented with 10% fetal bovine serum (FBS) and 1% penicillin/streptomycin solution. Cells were kept in an incubator at 37° C., under humidified atmosphere with 5% CO 2 for 2-3 days until confluence was reached. Next, cells were washed with Dulbecco's phosphate buffered saline solution (PBS—Thermo Fisher Scientific) and detached from the culture flasks using 0.05% trypsin/0.02% EDTA (Sigma). Cells were counted using a hemocytometer, then 10,000 cells per well were seeded in 96-well plates for 24 hours (n=6).

In order to obtain 1M stock solutions, each neat monomer used in the adhesive formulations: UDMA (urethane dimethacrylate), HEMA, DEBAAP, BMAAPMA, TMAAEA, and BAADA), was dissolved in dimethyl sulfoxide (DMSO). From these stocks, six different concentrated solutions (0.05, 0.1, 0.5, 1, 5 and 10 mM) were prepared in cell culture medium (DMEM for OD-21 and α-MEM for DPSC) and 100 μl per well was used to incubate the cells.

After cell exposure to the monomer solutions for 24 hours, 10 μl of 0.083% diphenyltetrazolium bromide (MTT) prepared in PBS (v/v) was added to each well and the cells were incubated for 4 hours. Next, 200 μl of DMSO was added to each well, plates were gently agitated and the optical density was read at 570 nm and 630 nm in a spectrophotometer (Epoch Microplate Spectrophotometer, BioTek Instruments Inc, Winooski, VT, USA). OD-21 and DPSCs cultured in the medium free from the monomers were used as negative controls. Additionally, since monomer fluorescence could affect the readings, 10 mM samples of each tested monomer were incubated without cells and their absorbance measured to provide a second control (i.e. medium control). The percentage of cell viability was calculated according to the equations:

OD_{cells}=(OD_{570 exper}−OD_{630 exper})−(OD_{570 med contr}−OD_{630 med contr}) Equation (3)

where: OD_{cell} is the absorbance of the remaining cells after exposure; ODₑₓₚₑᵣ is the absorbance of the experimental specimens at 570 nm and 630 nm; OD_{med contr} is the absorbance of the 10 mM solution of each monomer without cells at 570 nm and 630 nm.

$$CV ( \% ) = ( \frac{{OD}_{cells neg contr} - {OD}_{cells exper}}{{OD}_{cells neg contr}} ) \times 100 \qquad Equation ( 4 )$$

where: CV (%) is percentage of cell viability in relation to the negative control (cells+medium free from the monomers); OD_{cells neg contr} is the cells absorbance of the negative control; OD_{cells exper} is the cells absorbance of the experimental samples.

### Luciferase Assay and Impingement Test

A derivative of wild type UA159, the bioluminescent S. mutans strain IdhRenGSm [25] was selected for evaluating the antimicrobial potential of the adhesive resins. From a frozen stock, the bacteria was streaked onto an agar plate and grow out in an incubator at 37° C. under humidified atmosphere of 5% CO₂ in air for 1 day. At the end of this period, planktonic cultures were grown for 16 hours in TH culture medium supplemented with 10% yeast extract under the same conditions described above.

For antimicrobial activity evaluation, six discs (6 mm diameter×2 mm thick) were prepared in silicone molds, sandwiched between two glass slides covered with mylar strips, and photoactivated for 120 s on each side at 630 mW/cm². After 24 hours, the top surface was ground with 600-grit silicon-carbide paper in order to obtain a surface roughness standardized between 0.2 and 0.3 μm (Surftest Mitutoyo, cut-off length=0.25 mm, tracing speed=1.0 mm/s). Before incubation with the bacteria, the discs were sterilized in isopropyl alcohol for 20 minutes. It is important to note that the use of isopropanol has the potential to extract unreacted monomers from the specimens; therefore exposure time was limited to the minimum necessary to obtain sterile surfaces, as demonstrated in a previous study [26]. At the end of this period, specimens were rinsed vigorously to remove any remnant alcohol and then immersed in 48-well plates containing 1 ml of TH medium supplemented with 1% (w/v) sucrose and 1:500 dilution of the inoculum. One disc for each group was incubated in medium without inoculum as the sterility control. The plates were kept for 24 hours under 40 rpm agitation (Scilogex MX-M Microplate Mixer, Rocky Hill, CT, USA) in the CO₂ incubator.

The discs were then moved to 24-well black plates (Black Visiplate TC, Wallac, Finland) containing 0.5 ml of fresh TH medium per well and kept at 37° C. for 1 hour. Immediately, 5 μl of Coelenterazine-h ethanol solution was added to each well and the bioluminescence measured immediately by a spectrophotometer at 480 nm (GloMax Discover Multimode Microplate Reader, Promega Corporation). Details specific to the testing of the materials formulated with the quaternized monomers are described in the supplemental information.

### Statistical Analysis

Data were tested for normality (Anderson-Darling) and homocedasticity (Bartlett/Levene), then analyzed with one-way ANOVA and Tukey's test. Student's t-test was performed for comparison between storage conditions (dry versus wet). An overall significance level of α=0.05 was adopted for all tests.

### Results

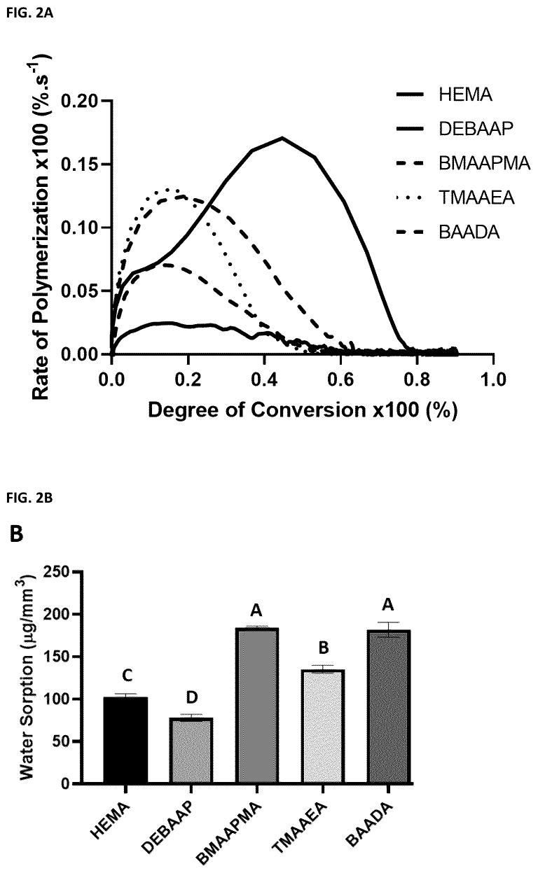

Results for degree of conversion, maximum rate of polymerization and conversion at rate maxima (used as a proxy for the onset of vitrification) values are shown in Table 1. Rate of polymerization as a function of degree of conversion for each of the formulations is shown in FIG. 2A. In general, the HEMA-containing formulation showed the highest values of final degree of conversion (DC) (90.4%), maximum rate of polymerization (Rpₘₐₓ) (17.1%·s⁻¹) and degree of conversion at maximum rate of polymerization (DC at Rpₘₐₓ) (44.7%). Among the tested acrylamides, TMAAEA formulation showed the lowest final DC (66.4%) and DEBAAP the lowest Rpₘₐₓ (2.4%·s⁻¹).

In terms of water sorption (WS), after 7 days water immersion, BMAAPMA and BAADA presented the highest values (184.3 and 181.9 μg/mm³, respectively) and DEBAAP the lowest ones (78.0 μg/mm³) (FIG. 2B). Solubility (SL) results ranged between −4.17 and 24.6 μg/mm³, with DEBAAP and BMAAPMA presenting the lowest values and BAADA the highest ones (FIG. 2C). In general, for both WS and SL, there was not a marked difference between methacrylate and acrylamide performance.

In relation to flexural strength (FS), there was a significant difference between the groups for both tested storage conditions (p<0.001). After 24 h dry storage, HEMA, DEBAAP and BMAAPMA formulation showed the highest results (127.9, 144.2, and 148.3 MPa, respectively) and TMAAEA the lowest ones (69.1 MPa). After 7 days water storage, all groups showed a decrease in FS, with DEBAAP showing the lowest reduction (24%), and HEMA, BMAAPMA, TMAAEA and BAADA formulations showing larger reductions of 37%, 60%, 64%, and 34%, respectively. A similar trend was seen for the elastic modulus (E) results, where HEMA, DEBAAP, BMAAPMA, and BAADA presented the highest results after dry storage (3.7, 3.5, 4.0, and 3.8 GPa, respectively) and TMAAEA the lowest in both storage conditions (2.0 and 0.7 GPa—dry and wet, respectively). DEBAAP was the most resistant formulation to the water storage and showed a reduction of only 11% in E. On the other hand, for HEMA, BMAAPMA, TMAAEA and BAADA more pronounced decreases were found (37%, 50%, 65% and 57%, respectively).

Dentin microtensile bond strength (MTBS) results are presented in Table 2. Significant differences were found among the materials for each storage period. At 24 hours, DEBAAP, TMAAEA and BAADA presented the highest results (33.0, 33.5 and 33.8 MPa, respectively) and HEMA the lowest (21.6 MPa). After 3-weeks storage, all groups showed comparable performance. After 6 months, TMAAEA and BAADA presented the highest results (36.4 and 32.5 MPa, respectively), and HEMA the lowest (12.4 MPa). With the exception of the HEMA-formulation, the MTBS within the same material was statistically similar for all storage periods (p<0.05). In general, a few of the newly synthesized acrylamides showed MTBS comparable to the commercial control at 24 h and 3-week storage times, but higher MTBS at 6 months.

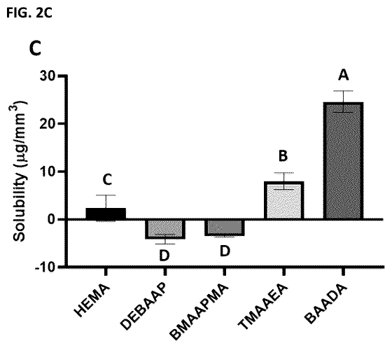

FIG. 3 shows a representative example of the NMR spectra and integration regions used to calculate monomer degradation. The proposed mechanism for the acid catalyzed hydrolysis of the different monomers is shown for 2-hydroxyethyl methacrylate (HEMA) in Figure S7 in the supplemental materials.

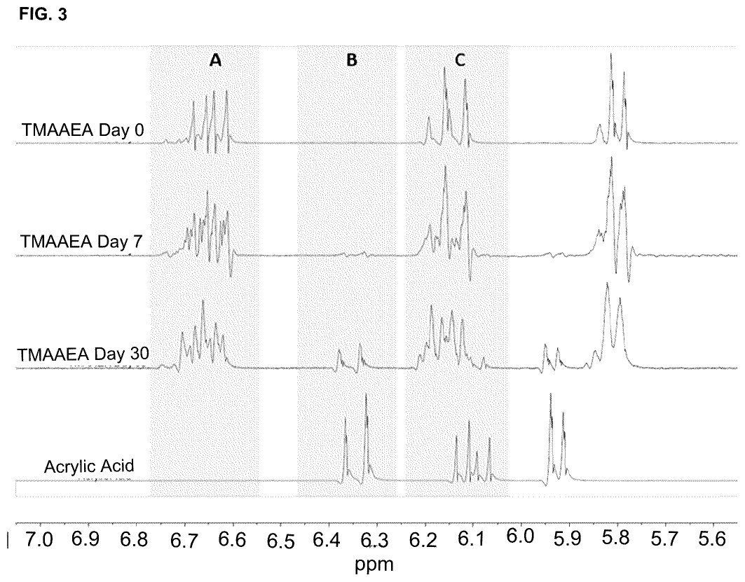

The degradation kinetics over one month as well as the final degradation percentage for each monomer in acidic aqueous conditions (pH=1) are shown in FIG. 4 and Table S1 (supplemental information). The methacrylate control HEMA degraded 89.38%, whereas the multifunctional acrylamides showed markedly less degradation, with TMAAEA degrading the most (6.29%). BMAAPMA, BAADA, and DEBAAP showed very little or no measurable degradation, averaging 1.27%, 1.13%, 0% respectively. HEMA and TMAAEA were fit to exponential decay curves to calculate monomer half-lives of 9.5 and 350 days.

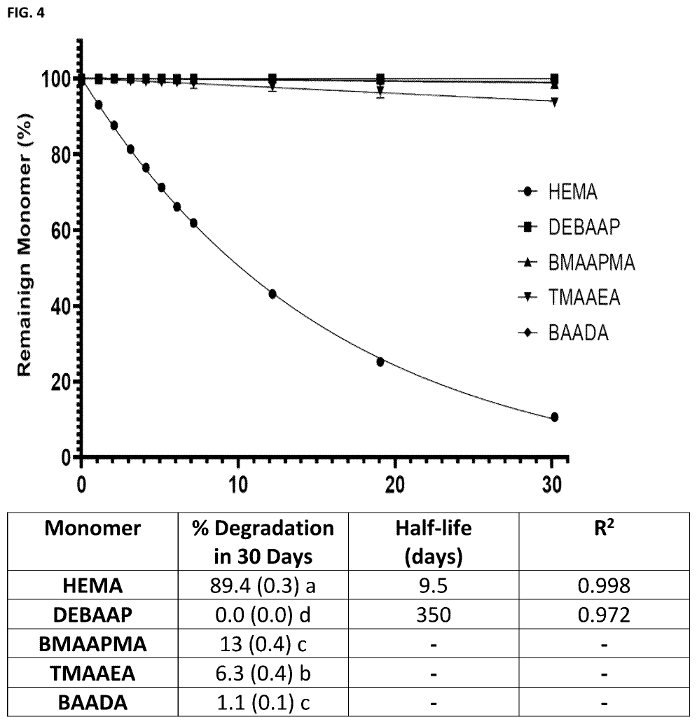

Cytotoxicity results are shown in FIG. 5. The percentage of cell viability for both cell lines decreased nearly monotonically with the increase in monomer concentration. Overall, for OD-21, the acrylamides showed biocompatibility comparable to UDMA and HEMA, with cell viability ranging between 41% and 59% in 10 mM concentration (FIG. 5A). The only exception was BMAAPMA, which showed 12% and 24% of remaining cells for 10 and 5 mM concentrations, respectively. The DPSC cell line was more sensitive to the monomers, and even in the lowest concentration the cell viability ranged between 59% and 86% (FIG. 5B).

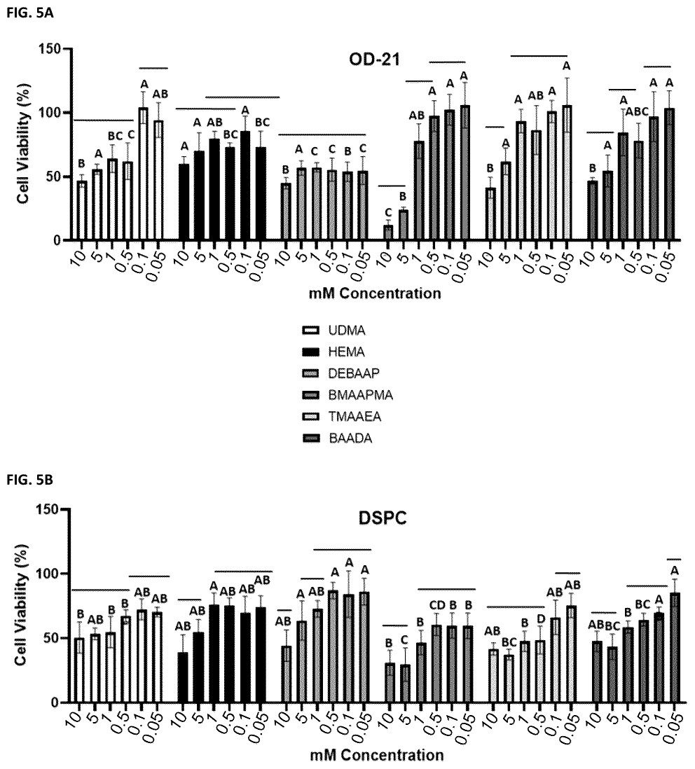

The Luciferase assay did not show any antimicrobial/antifouling effect for any of the non-quaternized monomer formulations tested (FIG. 6A). The relative light unit (RLU) values ranged between 2.80E+07 and 4.63E+07 and, despite the statistical difference (p<0.001), the values are within the same order of magnitude. The biofilm adhesion strength test did not show statistical difference among the groups, with only 1.4% being the highest percentage of biofilm totally removed from the surface among the tested groups.

### Discussion

Since the hydrolytic and the enzymatic degradation of the polymer are identified as contributors to the reduced service life of traditional methacrylate-based dental materials, this study designed, synthesized and tested polyacrylamides as alternative co-polymers for dental adhesive formulations. This approach has shown positive results in previous studies in terms of resistance to degradation, though in most cases, the accompanying reduction in mechanical properties precluded the use of those formulations in load-bearing applications such as restorative composites [27]. In this study, we focused on the use of multifunctional acrylamides that are liquids at room temperature as components of the dental adhesive, which is a topic that remains largely under-explored, although some recent reports demonstrate growing interest [28]. In general, multi-acrylamide formulations showed lower Rpₘₐₓ, DC at Rpₘₐₓ and final DC compared to the methacrylate control HEMA (FIG. 2A and Table 1). Despite its lower molecular weight and viscosity compared to the multi-functional acrylamides, the greater reactivity of HEMA was somewhat unexpected, since monofunctional monomers have demonstrated slower rates of polymerization compared to multi-functional counterparts, at least when sharing the same polymerizable functionality (i.e., methacrylates) [29]. In multi-methacrylates, the reaction of one vinyl bond reduces the likelihood of reaction of the other vinyl(s) on the same molecule, due to the diffusion limitation imposed by the attachment to the growing polymer chain [30]. In a study evaluating monofunctional monomers with systematically varied backbone structures, and including (meth)acrylates and (meth)acrylamides, the differences in reactivity were not as marked within the same polymerizing functionality, but were very obvious when comparing methacryl and acryl counterparts with analog backbones and substitutions [17]. In any case, monomethacrylates in general react slower because diffusional limitations that lead to auto-acceleration only occur much later in conversion—therefore, it is expected that monofunctional monomers achieve higher conversion, but at a lower rate [30]. Indeed, for HEMA, the rate maxima was observed at much higher conversion (close to 50%) compared to the acrylamides (around 15-19%), confirming that the onset of vitrification only happens later in conversion for the mono-functional monomer. The higher rate of polymerization for HEMA compared to the acrylamides is then strongly related to the greater reactivity of the methacrylate group compared to the resonance-stabilized acrylamide [31]. Indeed, among the multi-acrylamides, the trifunctional TMAAEA showed the highest maximum rate of polymerization and the lowest DC (66%), likely due to early gelation which led to autoacceleration earlier in the conversion [30]. The fact that the three functional groups are closely linked by short six-carbon arms, in association with the relatively high molecular weight (MW=350.46 g/mol), further contributes to the steric hindrance and diffusion limitations for this molecule.

Conversely, the di-functional DEBAAP showed high DC (74.4%), and markedly lower rate of polymerization (2.4%·s⁻¹). In comparison to the other tested compounds, in this molecule the two polymerizable functionalities are physically much closer together, which may cause the molecule to behave more like a mono-functional monomer, since access to the second vinyl would be more challenging once the first was anchored to the growing polymer chain. This slows down the propagation rate of the polymerization reaction. BAADA is also a short difunctional molecule, but it showed significantly higher reactivity (Rpₘₐₓ=7.3%·s⁻¹). This difference may be related to the seven-membered ring linker, which can assume two dominant conformations—envelope or twist [32]. These conformations reduce the steric strain and avoid the eclipse of C—C bonds, which potentially makes the C=C bonds more exposed to free radical attack compared to the linear DEBAAP [32]. BMAAPMA presented both high conversion and high rate of polymerization, likely due to the greater separation between the polymerizable functionalities through a flexible linker. In this case, even with the higher rate, the system likely maintained enough mobility to allow for further conversion to take place, which may indicate primary cyclization of the monomeric species. Thus, the conversion increases but without contributing to network formation [30].

When stored dry, all materials had flexural strengths and elastic moduli that were statistically similar to the control. The one exception was TMAAEA, which had significantly lower results, likely due to the reduced DC. After 7-days water storage, all materials had a reduction in flexural strength and modulus, ranging from 23-64% and 11-65%, respectively. With the exception of DEBAAP, which showed the highest flexural strength and elastic modulus after water storage, the acrylamides showed lower values and/or more significant reductions than the methacrylate control. This was expected due to the generally lower partition coefficient values (logP) for the amide-containing monomers. The logP determines the likelihood of a compound to partition into water or octanol, with the most hydrophilic compounds presenting lower logP values [33]. Amides are in general more hydrophilic than methacrylates because of the greater electronegativity of the nitrogen atom in the amide compared to the oxygen in the methacrylate. In addition, the presence of a lone pair of electrons on the nitrogen leads to stabilization of the carbon-nitrogen bond by resonance with the carbonyl [31]. The oxygen in the amide, in turn, assumes a partial negative charge (δ−), making it more prone to function as a hydrogen-bond acceptor. Conversely, the hydrogen of the N-H dipole is partially positively charged (δ+), which renders it a hydrogen-bond donor.

As result of these interactions, the amides are extremely prone to absorb and retain water [34]. In fact, the compounds with lower logP values in general led to greater water sorption and solubility results, partially explaining the drop in mechanical properties. The only reason why this relationship was not completely linear is the fact that TMAAEA presented lower WS/SL than BMAAPMA in spite of having lower logP. In this case, the fact that TMAAEA is trifunctional may have led to a more densely crosslinked network that was less prone to absorb water [35].

In the case of the diacrylamide DEBAAP, as mentioned, the mechanical properties after water storage were much less affected than any of the materials, including the methacrylate control, and showed a reduction of only 23% in flexural strength and 11% in modulus. In this case, it is likely that two factors are at play. The first is the high logP value (the highest among all groups). The second is the fact that the crosslink formed by DEBAAP is significantly shorter than any other of the multi-functional monomers tested, which likely led to a more densely packed network in the copolymerization with UDMA, in turn minimizing the plasticizing effect of water. In fact, others have demonstrated a linear relationship between the length of the crosslink and the free volume of the polymer network [36]. Both of these factors contributed to the lowest WS/SL results and the greatest network stability of DEBAAP-containing materials compared to all groups. Conversely, the other two polyacrylamides—BMAAPMA and TMAAEA presented a significant drop in mechanical properties (60 and 64% reduction on FS, respectively), which is likely due to the higher flexibility of these molecules and the less densely packed network formed after polymerization (for BMAAPMA), and the lower overall DC (for TMAAEA) in conjunction with the generally higher hydrophilicity of methacrylamides, as discussed previously. It is noteworthy that the solubility reflects the dissolution and leaching of various components, particularly unreacted monomers. For some of the groups, negative values of solubility resulted, which in practice means that the sample gained mass during the test. This rather unintuitive outcome is the result of some mass of the water being entrapped into the polymer network, and that mass being actually greater than the unreacted monomer mass lost. The ISO 4049 standard does not prescribe lyophilization of the samples to completely remove the entrapped water (it is important to highlight that the final mass (m3) is obtained after stabilization in a desiccator for at least 3 days). In any event, the negative values, in this case, mean that the solubility was very low.

All tested multi-acrylamides presented higher microtensile bond strength results at 24 h compared to the HEMA and commercial controls (the exception was BMAAPMA, which had statistically similar MTBS compared to the commercial control). In addition, all multi-acrylamides led to stable dentin bonding after 6-months storage, unlike the methacrylate experimental control (HEMA), which showed a 42% decrease over time. The bonding stability of the amides may be related, in part, to the high resistance to hydrolytic degradation (FIG. 4). Even though this was expected given the absence of ester groups and the strong resonance stabilization making the carbonyl carbon less polarized and consequently less susceptible to nucleophilic attack, the magnitude of the difference in performance of the traditional methacrylate diluent HEMA and the experimental multifunctional acrylamides was surprising. Of additional interest is the fact that the results suggest that the bond stability is not only a product of the material's mechanical properties, but is highly dependent on the quality of the interaction between the adhesive layer and the dental substrate. Several studies have shown that amides can form hydrogen-bond interactions with the carboxylic acids of the side-chain of aspartic and/or glutamic acids in the dentinal collagen [37-39]. In natural collagen, this leads to a triple helix structure formed via hydrogen bonding between the —C=O of the proline residue and the —NH of the glycine residue [37-39]. In theory, any amide could establish hydrogen bonds with collagen fibrils, which is likely to translate into stronger and more stable bond strength. In addition, other studies on cancer-targeting drugs have demonstrated that amides may potentially function as metalloproteinase (MMP) inhibitors [40, 41]. Even though the specific MMP enzymatic activity was not tested in this study, the more stable behavior of the amide-based adhesives compared to the methacrylate control, in the absence of significant differences in mechanical properties after wet storage, may be indicative that some effect at an enzymatic level is occurring. These aspects will be evaluated in detail in a separate investigation.

Dental adhesives intimately interact with vital dentine and, in many cases, are placed in close contact with the pulp chamber, which facilitates the diffusion of leachates and un-reacted monomers through the dentinal tubules into the pulp cavity at toxic concentrations [42]. It has been claimed that the release of certain adhesive components can cause genotoxic, allergic, cytotoxic, mutagenic, and estrogenic effects [42]. Despite the extensive use of acrylamides in wastewater treatment, general polymer industry, cosmetics and foods, they are identified as potentially cytotoxic for certain cell lines [43]. This is related to the fact that acrylamide functionality is highly electrophilic due to the presence of a lone pair of electrons in the nitrogen molecular orbital, which increases the susceptibility to reacting with biological nucleophilic molecules such as proteins and DNA, causing cell disruption [44, 45]. Thus, the biocompatibility of the compounds was evaluated with undifferentiated pulp cells (OD-21) and human dental pulp stem cells (h-DPSC).

OD-21 is an immortalized cell line genetically engineered from mouse models, which are a reliable source for screening and repeatable tests due to their homogeneous morphology and growth characteristics [46]. On the other hand, h-DSPC is a finite life span cell line that plays a crucial role on human pulp repair process, migrating toward the injury site and differentiating into odontoblast-like cells to produce reparative dentin [47, 48]. The results showed that h-DPSC cells were more sensitive to the presence of monomers than OD-21, which resulted in lower percentages of cell viability (FIG. 5B). This was expected as the immortalized cells are more robust due to their ability to continually undergo cell division. Significant differences were found between the monomers at higher mM concentrations for the OD-21 cell line. At 10 and 5 mM, BMAAPMA showed the lowest cell viability, HEMA the highest and UDMA, DEBAAP, TMAAEA and BAADA with similar intermediate results. Interestingly, at lower mM concentrations, the novel designed multi-acrylamides were more biocompatible than HEMA. These results were somewhat unexpected due to the reported cytotoxicity of some acrylamides. It has been shown that acrylamides can form adducts with the reduced glutathione (an important antioxidant—GSH) and increase the production of hydrogen peroxide, which may increase the level of lipid peroxidation (LPO) and carbonyl content, and decrease the enzymatic and non-enzymatic antioxidant activity [49, 50]. In summary, this makes the cells more vulnerable to free radical damage. In addition, amides can bind to sulfhydryl groups to inactivate proteins/enzymes involved in DNA repair and other critical cell functions [49]. Since the toxic mechanisms involve the interaction with the amide functionality site, it is expected that the more flexible BMAAPMA would be more toxic. Also, for analogue monomers bearing methacryl or acryl functionalities, the presence of the methyl group in the former is expected to render the molecule less prone to nucleophilic attack, and therefore, less likely to disturb cellular anti-oxidative mechanisms. However, the comparison between methacrylates and acrylamides is not as straightforward as that between methacrylates and acrylates, for example. Methacrylates are less susceptible to nucleophilic attack in comparison to acrylates for the reasons already mentioned. However, for acrylamides, particularly tertiary acrylamides such as those used in this study, resonance stabilization in the amide carbonyl renders this molecule more stable than the ester-containing methacrylate, as shown by the degradation data (FIG. 4). In addition, compared with the mono-functional HEMA, the tertiary acrylamides used here had higher molecular weight (about 2-3 fold higher) and greater degree of functionality (di- or tri-functional), which may have partially prevented their extraction to the culture medium. Indeed, the hydrolytic degradation data shows that the concentration of intact HEMA significantly decreased in the first 24 h, so it is reasonable to assume that the presence of methacrylic acid, a by-product of ester degradation, significantly contributed to the biological toxicity of this group [51]. Based on that and the similarity between the tested concentrations, it is possible to assume that, despite not being lethal at high concentrations, even small amounts of HEMA can irreversibly injure the cells.

DPSC results showed a different trend. In general, acrylamides and methacrylates showed similar results with few exceptions: [5 mM] BMAAPMA and [0.5 mM] TMAAEA were less biocompatible than the tested methacrylates. Since DPSC are highly sensitive, methacrylates and acrylamides were equally able to damage the cells, though likely through different mechanisms, overshadowing differences between the compounds such as the higher electrophilicity of the amide functionality in comparison with the ester group. In general, all monomers exhibited dose-dependent cytotoxic effects, which was expected [14]. However, at the highest concentrations—10 and 5 mM, for all tested groups, the monomers induced notable cytotoxic effects, but with no statistical difference between them. This may indicate that there is a threshold in concentration above which the cells become saturated, and no additional cytotoxicity is recorded with the methods used here.

It is important to highlight that the neat monomers were tested instead of the copolymers in order to evaluate the potential toxicity of the leached unreacted monomers. The leaching process is dependent on polymer degree of conversion, molecular weight and hydrophilicity of the monomer, and extraction solvent [52]. One previous study has shown that hydrophilic low-molecular weight HEMA can be released from polymerized dental adhesives from 1.5 to 8 mM [53], while the highest amount of the hydrophobic high-molecular weight BisGMA was 425 μg/ml (0.83 mM) from an orthodontic adhesive [54]. Therefore, it is difficult to predict a clinically-relevant concentration of leached monomers for the novel multi-acrylamides, but it is possible to assume that they will behave closer to BisGMA than HEMA and, at these concentrations, the results of the present study indicate that the amides and the traditional methacrylates are equally biocompatible.

The upregulating role of some dental monomers on the growth and proliferation of microorganisms involved in the caries development process has been demonstrated [55, 56]. Among other effects, these monomers can also potentially increase the activity of glycosyltransferase, which would increase bacterial extra-cellular matrix formation, therefore maximizing bacterial adhesion and mature biofilm formation [57-59]. In this study, the alternative adhesive formulations were subjected to a biofilm growth assay, based on a previously described renilla-reporter method [25]. Overall, the results showed that the multi-acrylamide formulations were statistically similar to the control group (FIG. 6A). The only exception was BAADA, which showed significantly higher values, though still within the same order of magnitude. BAADA is a solid powder and did not form a homogenous mixture with UDMA, which can potentially explain this difference. It is possible that some level of porosity (not noticeable with the naked eye) would provide a favorable surface for S. mutans growth. This argument, however, is somewhat negated by the fact that the initial surface roughness of the discs had been standardized before incubation. Interestingly, this slight increase in the biofilm growth did not translate into stronger biofilm adhesion as measured by the impingement test, which was similar to other tested groups.

In summary, this work was focused on the maintenance of the bond strength over time, and tested selected formulations for potential antimicrobial activity. From that standpoint, BMAAPMA, TMAAEA and BAADA stood out. The control (HEMA) showed more than 40% reduction in bond strength after 6 months, while those monomers were able to maintain the initial results. This was true in spite of the drop in mechanical properties. The antimicrobial activity of these materials was assessed in this study to provide a thorough evaluation, and to ensure that there was no obvious up or down regulating effect on biofilm formation—and that was confirmed. Several other applications where adhesion to mineralized tissues are required can potentially benefit from this technology, since the polyacrylamides already in use present several drawbacks [60].

### Conclusion

The incorporation of the newly synthesized multi-acrylamides in dental adhesive formulations led to higher and more stable dentin bonding strength with marked resistance to hydrolytic degradation, representing a suitable alternative to the ester containing monomers. Screening biocompatibility, biofilm formation and the other assessed properties did not indicate limiting issues for the clinical applicability.

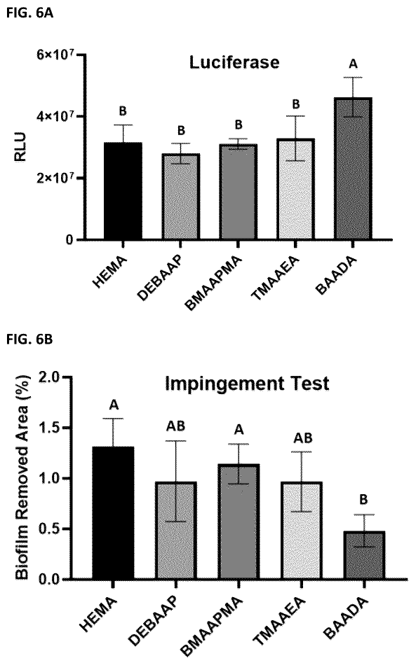

Table 1 (FIG. 7) presents mean and standard deviation of degree of conversion (DC in %), maximum rate of polymerization (Rpmax in %·s−1), DC at Rpmax (in %), flexural strength (FS, MPa), and elastic modulus (E, GPa). Values followed by the same lowercase letter on the same column and uppercase letter on the same row are statistically similar. The one-way ANOVA probability values (p) for each assessed experimental parameter are depicted on the last line.

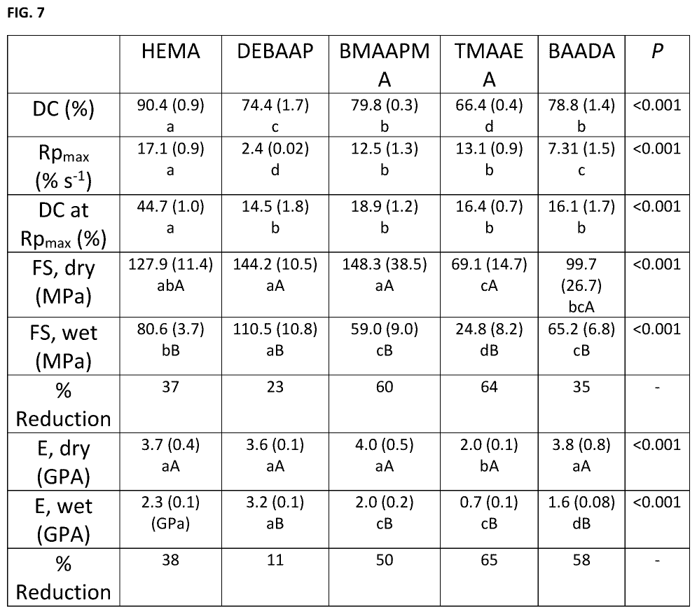

Table 2 (FIG. 8) presents mean and standard deviation of microtensile bond strength tests (MPa) for adhesive formulations after storage in distilled water at 37° C. for 24 hours, 3 weeks and 6 months. Values followed by the same lowercase letter in the same column are statistically similar, and values followed by the same uppercase letter in the same row are statistically similar. The one-way ANOVA probability values of the comparison among the groups within the same storage time (p) and the comparison among the storage times within the same experimental group (p*) are presented.

### Synthesis of Quaternary Diacrylamides

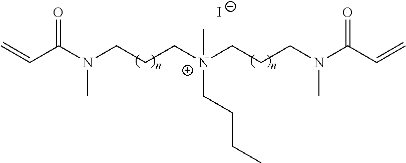

N-methyl-N,N-Bis[(3-methylaminoacryl)propyl]butan-1-aminium iodide (BMAAPMA-C4) or N-butyl-N -methyl-6-(methylamino)-N-(6-(methylamino)-4-oxohex-5-en-1-yl)-4-oxohex-5-en-1-aminium iodide: BMAAPMA (6.35 mmol, 1.79 g) and MEHQ (2.0 mg) were dissolved in 5 mL of acetone. 1-iodobutane (5 mL) was added and the mixture was heated to 50° C. and left to stir for 5 days. After removal from heat, the biphasic mixture settled to form a dark orange top layer (25%) and a light orange bottom layer (75%). The top layer was removed and a precipitated into 20 mL of ether to form a viscous orange oil. The ether was decanted, and the oil was triturated twice more with ether. Excess solvent was removed in vacuo to yield 2.20 g of a viscous orange solid (4.71 mmol, 74.2% yield). ¹H NMR (400 MHz, Deuterium Oxide) δ 6.76-6.53 (m, 2H), 6.23-6.02 (m, 2H), 5.89-5.63 (m, 2H), 3.59-3.37 (m, 4H), 3.28-3.16 (m, 6H), 3.07 (s, 6H), 2.93 (s, 3H), 2.09-1.87 (m, 4H), 1.68-1.52 (m, 2H), 1.29 (q, J=7.5 Hz, 2H), 0.87 (t, J=7.4 Hz, 3H).

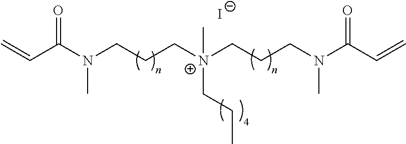

N-methyl-N,N-Bis[(3-methylaminoacryl)propyl]hexan-1-aminium iodide (BMAAPMA-C6) or N-hexyl-N-methyl-6-(methylamino)-N-(6-(methylamino)-4-oxohex-5-en-1-yl)-4-oxohex-5-en-1-aminium iodide: BMAAPMA (6.34 mmol, 1.78 g) and MEHQ (2.0 mg) were dissolved in 5 mL of acetone. 1-iodohexane (5 mL) was added and the mixture was heated to 50° C. and left to stir for 5 days. After removal from heat, the biphasic mixture settled to form a dark orange top layer (25%) and a light orange bottom layer (75%). The top layer was removed and a precipitated into 20 mL of ether to form a viscous orange oil. The ether was decanted, and the oil was triturated twice more with ether. Excess solvent was removed in vacuo to yield 2.49 g of a viscous orange solid (5.04 mmol, 79.5% yield). ¹H NMR (400 MHz, Deuterium Oxide) δ 6.73-6.55 (m, 2H), 6.22-6.00 (m, 2H), 5.83-5.67 (m, 2H), 3.59-3.35 (m, 4H), 3.21 (s, 6H), 3.08 (s, 6H), 3.00-2.84 (m, 3H), 2.09-1.86 (m, 4H), 1.60-1.50 (m, 2H), 1.36-1.14 (m, 6H), 0.80 (d, J=7.2 Hz, 3H).

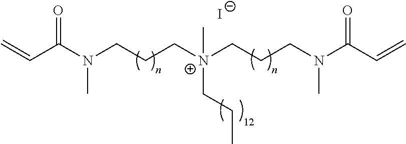

N-tetradecyl-N,N-Bis[(3-methylaminoacryl)propyl]tetradecan-l-aminium iodide (BMAAPMA-C14) or N -methyl-N,N-bis(6-(methylamino)-4-oxohex-5-en-1-yl)tetradecan-1-aminium iodide: BMAAPMA (4.62 mmol, 1.30 g) and MEHQ (1.5 mg) were dissolved in 10 mL of acetone. 1-iodohexane (5 mL) was added and the mixture was heated to 65° C. and left to stir for 5 days. After removal from heat, the biphasic mixture settled to form a dark orange top layer (25%) and a light orange bottom layer (75%). The top layer was removed and a precipitated into 20 mL of ether to form a viscous orange oil. The ether was decanted, and the oil was triturated twice more with ether. Excess solvent was removed in vacuo to yield 1.67 g of a viscous orange solid (2.75 mmol, 59.6% yield). ¹H NMR (400 MHz, Deuterium Oxide) δ 6.80-6.56 (m, 2H), 6.21-6.03 (m, 2H), 5.78-5.63 (m, 2H), 3.60-3.38 (m, 4H), 3.38-3.15 (m, 6H), 3.05 (s, 6H), 2.93 (s, 3H), 2.11-1.82 (m, 4H), 1.74-1.46 (m, 2H), 1.40-1.05 (m, 22H), 0.82 (t, J=5.7 Hz, 3H).

### Acid-Catalyzed Hydrolysis Mechanism

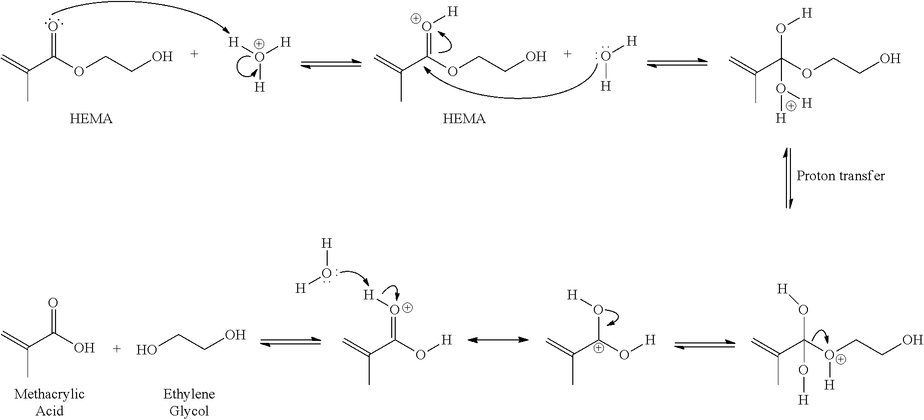

Scheme above: An example of 2-hydroxyethyl methacrylate (HEMA) hydrolysis to methacrylic acid and ethylene glycol in acidic conditions to show the mechanism of acid catalyzed hydrolysis of (meth)acrylate and (meth)acrylamides ester bonds (Grossman RB. 2003. The art of writing reasonable organic reaction mechanisms. New York: Springer-Verlag).

Monomer degradation as a function of time (average and standard deviation) as assessed by H-NMR.

| | Percent remaining monomer | | | | |
|---|---|---|---|---|---|
| Days | HEMA | DEBAAP | BMAAPMA | TMAAEA | BAADA |
| 0 | 99.2 ± 0.6 | 100.0 ± 0.0 | 100.0 ± 0.0 | 100.0 ± 0.0 | 100.0 ± 0.0 |
| 1 | 93.1 ± 0.04 | 100.0 ± 0.0 | 99.8 ± 0.01 | 99.8 ± 0.2 | 100.0 ± 0.0 |
| 2 | 87.6 ± 0.02 | 100.0 ± 0.0 | 100.0 ± 0.0 | 99.8 ± 0.2 | 100.0 ± 0.0 |
| 3 | 81.3 ± 0.02 | 100.0 ± 0.0 | 100.0 ± 0.0 | 99.3 ± 0.7 | 100.0 ± 0.0 |
| 4 | 76.5 ± 0.01 | 100.0 ± 0.0 | 100.0 ± 0.0 | 99.2 ± 0.9 | 99.8 ± 0.2 |
| 5 | 71.3 ± 0.02 | 100.0 ± 0.0 | 99.7 ± 0.01 | 99.1 ± 0.7 | 99.9 ± 0.1 |
| 6 | 66.1 ± 0.02 | 99.9 ± 0.2 | 100.0 ± 0.0 | 99.0 ± 1.0 | 100.0 ± 0.0 |
| 7 | 61.9 ± 0.01 | 100.0 ± 0.0 | 100.0 ± 0.0 | 98.7 ± 1.3 | 99.9 ± 0.1 |
| 12 | 43.1 ± 0.02 | 100.0 ± 0.0 | 100.0 ± 0.0 | 997.9 ± 1.2 | 99.6 ± 0.4 |
| 19 | 25.2 ± 0.02 | 100.0 ± 0.0 | 99.7 ± 0.3 | 96.7 ± 1.8 | 92.3 ± 0.7 |
| 30 | 10.6 ± 0.1 | 100.0 ± 0.0 | 98.7 ± 1.3 | 93.7 ± 0.7 | 98.9 ± 1.2 |

### Detailed Plot for Water Sorption and Solubiltiy

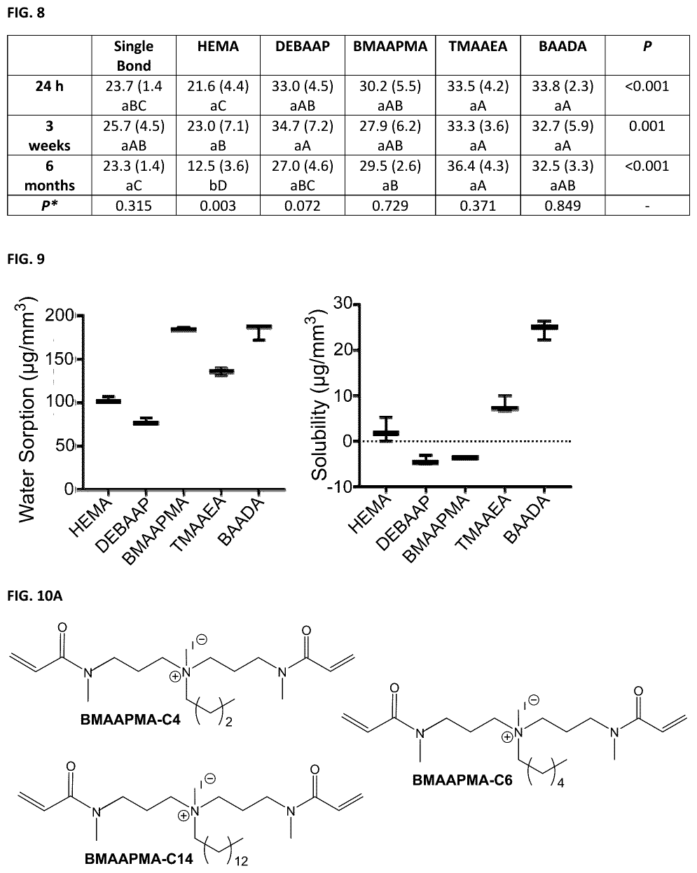

FIG. 9 provides box plot graphs of water sorption and solubility (μg/mm³) for tested formulations.

### Half Maximal Inhibitory Concentration (IC₅₀) and Derived pKa

The half maximal inhibitory concentration (IC₅₀) of each tested monomer was determined based on the plots of cell inhibition (%) as a function of the monomers concentration (mM) using GraphPad software.

The values for pKa for each tested monomer was predicted by ChemDraw Professional software.

The half maximal inhibitory concentration (IC50) (mM) of all tested neat monomers for odontoblast-like cell line (OD-21) and human dental pulp stem cells (DPSC) and their respective derived pKa values.

| | IC₅₀ | | |
|---|---|---|---|
| Monomer | OD-21[mM] | DPSC [mM] | Derived pKa |
| UDMA | 7.94 | 10.26 | — |
| HEMA | 15.39 | 6.87 | 13.942 |
| DEBAAP | 7.11 | 8.43 | na |
| BMAAPMA | 4.75 | 0.54 | 9.284 |
| TMAAEA | 8.02 | 1.07 | 7.188 |
| BAADA | 9.98 | 3.65 | na |

* The IC50 values highlighted in bold indicate logarithmic trend. The other groups showed linear trend.

### Quaternization of Tertiary Acrylamides for Added Antimicrobial Activity

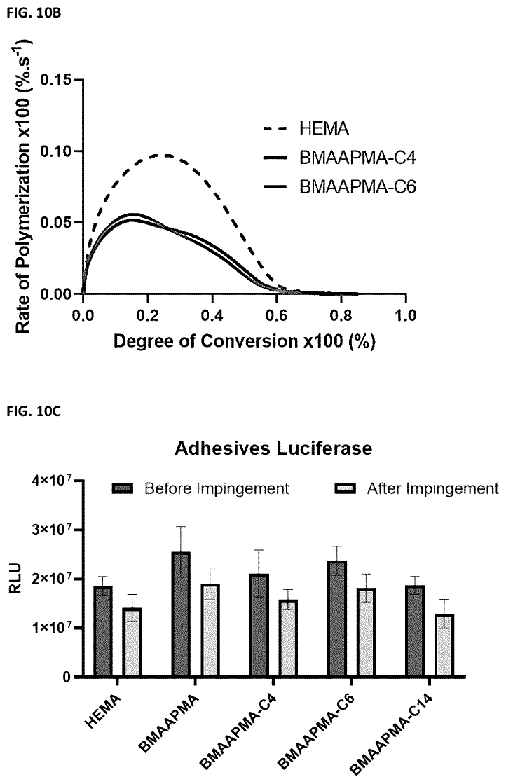

Given the extensive effort dedicated to produce resin monomers with antimicrobial properties to, in turn, minimize the occurrence of secondary caries, the bifunctional acrylamide BMAAPMA was quaternized with substituents of three chain lengths (4, 6 and 14 carbons) to form quaternary ammonium acrylamides. These were used to replace part of the non-quaternized BMAAPMA in the UDMA-based organic matrix. The biofilm adhesion strength was evaluated immediately after the bioluminescence measurements using the microjet impingement method (Kreth et al., Biofilms. 1(4):277-284). For this assay, a 0.2-mm ID nozzle was placed perpendicular to the discs at a distance of 0.4 mm and a water stream was dispensed for 5 s at 45 psi. Remaining biofilms were then stained with crystal violet (10% aqueous solution for 2 min) and imaged with a light microscope. The free biofilm area was measured by tracing with Image J software and the loss of adhesion (in percentage) was determined for each disc. For the quaternized monomers used in the additional formulations (BMAAPMA-C4, C6 and C14), instead of the crystal violet staining, the luciferase assay was repeated after impingement. This test was done for these monomers (and validated for HEMA and non-quaternized BMAAPMA controls) to assess their antimicrobial activity. The crystal violet method was not used in this case because, rather than test for a change in adhesion, we were assessing a potential true bactericidal effect on the bacteria that would minimize or eliminate biofilm production. FIG. 10 shows the results of polymerization kinetics and antimicrobial activity for the quaternized monomers. Structures are shown in FIG. 10A. Final degree of conversion was similar for all monomers tested (p>0.05, HEMA=84.1±1.3%, BMAAPMA-C4=85.3±3.2%, and BMAAPMA-C6=84.5±2.6%), but the Rpₘₐₓ was lower for the acrylamides compared with the HEMA control (FIG. 10B). The luciferase activity was similar for all groups, before and after impingement (FIG. 10C). The kinetics results demonstrated lower Rpₘₐₓ in relation to the HEMA control, but similar final conversion, as expected based on previous results (Fugolin et al. Acta Biomaterialia 100 (2019) 132-141) and according to what was also observed for the non-quaternized monomers. In terms of antimicrobial activity, surprisingly, the quaternized monomer did not show significant effects, which is a strong indication of the key role played by chemical structure on the interaction with microorganisms. Charge distribution and steric issues might be possible causes for the lack of interaction between the compound and S. mutans.

In FIG. 10. (A) Chemical structures of the diacrylamides quaternized with 4, 6 and 14 chain length carbons. These compounds were added in 10 wt % in formulations containing UDMA and BMAAPMA (60:30) and tested for kinetics and biofilm formation. (B) Rate of polymerization (%·s⁻¹) as a function of degree of conversion (%) curves showed significantly lower reactivity of the quaternized compounds, which was not translated in lower final degree of conversion. (C) Luciferase results before and after biofilm adhesion test did not highlight significant antimicrobial activity of the novel compounds.

### REFERENCES

[1] A. Frassetto, L. Breschi, G. Turco, G. Marchesi, R. Di Lenarda, F. R. Tay, D. H. Pashley, M. Cadenaro, Mechanisms of degradation of the hybrid layer in adhesive dentistry and therapeutic agents to improve bond durability—A literature review, Dental Materials 32(2) (2016) e41-e53.
[2] L. Tjäderhane, H. Larjava, T. Sorsa, V. J. Uitto, M. Larmas, T. Salo, The activation and function of host matrix metalloproteinases in dentin matrix breakdown in caries lesions, Journal of Dental Research 77(8) (1998) 1622-1629.
[3] A. Mazzoni, F. D. Nascimento, M. Carrilho, I. Tersariol, V. Papa, L. Tjäderhane, R. Di Lenarda, F. R. Tay, D. H. Pashley, L. Breschi, MMP activity in the hybrid layer detected with in situ zymography, Journal of Dental Research 91(5) (2012) 467-472.
[4] I. L. Tersariol, S. Geraldeli, C. L. Minciotti, F. D. Nascimento, V. Pääkkönen, M. T. Martins, M. R. Carrilho, D. H. Pashley, F. R. Tay, T. Salo, L. Tjäderhane, Cysteine Cathepsins in Human Dentin-Pulp Complex, Journal of Endodontics 36(3) (2010) 475-481.
[5] F. D. Nascimento, C. L. Minciotti, S. Geraldeli, M. R. Carrilho, D. H. Pashley, F. R. Tay, H. B. Nader, T. Salo, L. Tjäderhane, I.L.S. Tersariol, Cysteine cathepsins in human carious dentin, Journal of Dental Research 90(4) (2011) 506-511.
[6] X. Wang, S. Song, L. Chen, C. M. Stafford, J. Sun, Short-time dental resin biostability and kinetics of enzymatic degradation, Acta Biomaterialia 74 (2018) 326-333.
[7] Y. Finer, J. Santerre, Biodegradation of a dental composite by esterases: dependence on enzyme concentration and specificity, Journal of Biomaterials Science, Polymer Edition 14(8) (2003) 837-849.
[8] Y. Finer, J. P. Santerre, Salivary esterase activity and its association with the biodegradation of dental composites, Journal of Dental Research 83(1) (2004) 22-26.
[9] Y. Finer, F. Jaffer, J. P. Santerre, Mutual influence of cholesterol esterase and pseudocholinesterase on the biodegradation of dental composites, Biomaterials 25(10) (2004) 1787-1793.
[10] F. Abedin, Q. Ye, R. Parthasarathy, A. Misra, P. Spencer, Polymerization Behavior of Hydrophilic-Rich Phase of Dentin Adhesive, Journal of Dental Research 94(3) (2015) 500-507.
[11] P. Spencer, Y. Wang, Adhesive phase separation at the dentin interface under wet bonding conditions, Journal of Biomedical Materials Research: An Official Journal of The Society for Biomaterials, The Japanese Society for Biomaterials, and The Australian Society for Biomaterials and the Korean Society for Biomaterials 62(3) (2002) 447-456.
[12] J. G. Park, Q. Ye, E. M. Topp, C. H. Lee, E. L. Kostoryz, A. Misra, P. Spencer, Dynamic mechanical analysis and esterase degradation of dentin adhesives containing a branched methacrylate, Journal of Biomedical Materials Research—Part B Applied Biomaterials 91(1) (2009) 61-70.
[13] E. Morisbak, S. Uvsløkk, J. T. Samuelsen, In vitro effects of dental monomer exposure—Dependence on the cell culture model, Toxicology in Vitro 67 (2020).
[14] A. Bakopoulou, T. Papadopoulos, P. Garefis, Molecular toxicology of substances released from resin-based dental restorative materials, International Journal of Molecular Sciences 10(9) (2009) 3861-3899.
[15] M. MacAulay, L. E. Tam, J. P. Santerre, Y. Finer, In Vivo Biodegradation of bisGMA and Urethane-Modified bisGMA-Based Resin Composite Materials, JDR Clinical & Translational Research 2(4) (2017) 397-405.
[16] R. Becher, H. Wellendorf, A. K. Sakhi, J. T. Samuelsen, C. Thomsen, A. K. Bølling, H. M. Kopperud, Presence and leaching of bisphenol a (BPA) from dental materials, Acta Biomater Odontol Scand 4(1) (2018) 56-62.
[17] A. P. Fugolin, A. Dobson, W. Mbiya, O. Navarro, J. L. Ferracane, C. S. Pfeifer, Use of (meth)acrylamides as alternative monomers in dental adhesive systems, Dental Materials (2019).
[18] N. Moszner, F. Zeuner, J. Angermann, U. K. Fischer, V. Rheinberger, Monomers for adhesive polymers, 4: Synthesis and radical polymerization of hydrolytically stable crosslinking monomers, Macromolecular Materials and Engineering 288(8) (2003) 621-628.
[19] A. P. Fugolin, A. Dobson, V. Huynh, W. Mbiya, O. Navarro, C. M. Franca, M. Logan, J. L. Merritt, J. L. Ferracane, C. S. Pfeifer, Antibacterial, Ester-Free Monomers: Polymerization Kinetics, Mechanical Properties, Biocompatibility and Anti-Biofilm Activity, Acta Biomaterialia (2019).
[20] K. Zhang, L. Cheng, M.D. Weir, Y. X. Bai, H. H. Xu, Effects of quaternary ammonium chain length on the antibacterial and remineralizing effects of a calcium phosphate nanocomposite, International journal of oral science 8(1) (2016) 45-53.
[21] J. W. Stansbury, S. H. Dickens, Determination of double bond conversion in dental resins by near infrared spectroscopy, Dental Materials 17(1) (2001) 71-79.
[22] I.S. 4049, ISO 4049 polymer based filling, restorative and luting materials, International Organization for Standardization 1 (2000) 27.
[23] M. ESPE, 3M ESPE ADPER SINGLE BOND 2, 2020. http://multimedia.3m.com/mws/mediawebserver?mwsld=SSSSSuUn zu8l00x4YtGlx210v70k17zHvu9lxt D7SSSSSS--. (Accessed 02/26 2020).
[24] L. M. Barcelos, M. G. Borges, C. J. Soares, M. S. Menezes, V. Huynh, M. G. Logan, A. P. P. Fugolin, C. S. Pfeifer, Effect of the photoinitiator system on the polymerization of secondary methacrylamides of systematically varied structure for dental adhesive applications, Dental Materials 36(3) (2020) 468-477.
[25] J. Merritt, J. Kreth, F. Qi, R. Sullivan, W. Shi, Non-disruptive, real-time analyses of the metabolic status and viability of Streptococcus mutans cells in response to antimicrobial treatments, Journal of Microbiological Methods 61(2) (2005) 161-170.
[26] C. B. André, A. dos Santos, C. S. Pfeifer, M. Giannini, E. M. Girotto, J. L. Ferracane, Evaluation of three different decontamination techniques on biofilm formation, and on physical and chemical properties of resin composites, Journal of Biomedical Materials Research—Part B Applied Biomaterials 106(3) (2018) 945-953.
[27] N. Moszner, U. K. Fischer, J. Angermann, V. Rheinberger, Bis-(acrylamide)s as new cross-linkers for resin-based composite restoratives, Dental Materials 22(12) (2006) 1157-1162.
[28] S. B. Rodrigues, C. L. Petzhold, D. Gamba, V. C. B. Leitune, F. M. Collares, Acrylamides and methacrylamides as alternative monomers for dental adhesives, Dental Materials 34(11) (2018) 1634-1644.
[29] F. M. Collares, F. A. Ogliari, C. H. Zanchi, C. L. Petzhold, E. Piva, S. M. W. Samuel, Influence of 2-Hydroxyethyl Methacrylate Concentration on Polymer Network of Adhesive Resin, The Journal of Adhesive Dentistry 13(2) (2011) 125-129.
[30] G. Odian, Principles of polymerization, John Wiley & Sons2004.
[31] M. Tian, Y. Xu, Monomer reactivity ratios of acrylamide and 2-ethylhexyl acrylate determined by the elemental analysis method, Oxidation Communications 39(4) (2016) 3357-3369.
[32] T. Soderberg, Organic chemistry with a biological emphasis, Timothy Soderberg2012.
[33] S. H. Dickens, G. M. Flaim, C. J. E. Floyd, Effects of adhesive, base and diluent monomers on water sorption and conversion of experimental resins, Dental Materials 26(7) (2010) 675-681.
[34] J. DeRuiter, Principles of drug action 1- Amides and Related Functional Groups, Spring2005.
[35] J. -G. Park, Q. Ye, E. M. Topp, A. Misra, P. Spencer, Water sorption and dynamic mechanical properties of dentin adhesives with a urethane-based multifunctional methacrylate monomer, Dent Mater 25(12) (2009) 1569-1575.
[36] K. S. Anseth, C. N. Bowman, Kinetic Gelation model predictions of crosslinked polymer network microstructure, Chemical Engineering Science 49(14) (1994) 2207-2217.
[37] N. Nishiyama, T. Asakura, K. Suzuki, K. Komatsu, K. Nemoto, Bond Strength of Resin to Acid-etched Dentin Studied by 13C NMR: Interaction between N-methacryloyl-w-Amino Acid Primer and Dentinal Collagen, Journal of Dental Research 79(3) (2000) 806-811.
[38] N. Nishiyama, T. Asakura, K. Suzuki, T. Sato, K. Nemoto, Adhesion mechanisms of resin to etched dentin primed with N-methacryloyl glycine studied by 13C-NMR, Journal of Biomedical Materials Research 40(3) (1998) 458-463.
[39] N. Nishiyama, K. Suzuki, T. Asakura, K. Komatsu, K. Nemoto, Adhesion of N-methacryloyl-w-amino acid primers to collagen analyzed by 13C NMR, Journal of Dental Research 80(3) (2001) 855-859.
[40] Z. H. Shi, N. G. Li, Q. P. Shi, H. Tang, Y. P. Tang, W. Li, L. Yin, J. P. Yang, J. A. Duan, Design, synthesis and biological evaluation of ferulic acid amides as selective matrix metalloproteinase inhibitors, Medicinal Chemistry 9(7) (2013) 947-954.
[41] C. P. Decicco, J. L. Seng, K. E. Kennedy, M. B. Covington, P. K. Welch, E. C. Arner, R. L. Magolda, D. J. Nelson, Amide surrogates of matrix metalloproteinase inhibitors: Urea and sulfonamide mimics, Bioorganic & Medicinal Chemistry Letters 7(18) (1997) 2331-2336.
[42] A. Tadin, L. Gavić, Biocompatibility of Dental Adhesives, in: A. Rudawska (Ed.), Adhesives-Applications and Properties, IntechOpen2016, p. 915.
[43] S. Kacar, D. Vejselova, H. M. Kutlu, V. Sahinturk, Acrylamide-derived cytotoxic, anti-proliferative, and apoptotic effects on A549 cells, Human and Experimental Toxicology 37(5) (2018) 468-474.
[44] P. T. Anastas, Green processes: designing safer chemicals, Wiley Interscience, Hoboken, NJ, 2013.
[45] J. B. Zimmerman, P. T. Anastas, Toward designing safer chemicals, Science 347(6219) (2015) 215.
[46] C. T. Hanks, Z. L. Sun, D. N. Fang, C. A. Edwards, J. C. Wataha, H. H. Ritchie, W. T. Butler, Cloned 3T6 cell line from CD-1 mouse fetal molar dental papillae, Connective Tissue Research 37(3-4) (1998) 233-249.
[47] A. Bakopoulou, G. Leyhausen, J. Volk, E. Papachristou, P. Koidis, W. Geurtsen, Wnt/β-catenin signaling regulates Dental Pulp Stem Cells' responses to pulp injury by resinous monomers, Dental Materials 31(5) (2015) 542-555.
[48] I. About, Dentin-pulp regeneration: the primordial role of the microenvironment and its modification by traumatic injuries and bioactive materials, Endodontic Topics 28(1) (2013) 61-89.
[49] J. Exon, A review of the toxicology of acrylamide, J Toxicol Environ Health B Crit Rev 9(5) (2006) 397-412.
[50] G. Song, Z. Liu, Q. Liu, X. Liu, Lipoic acid prevents acrylamide-induced neurotoxicity in CD-1 mice and BV2 microglial cells via maintaining redox homeostasis, Journal of Functional Foods 35 (2017) 363-375.
[51] S. Kurata, K. Morishita, T. Kawase, K. Umemoto, Cytotoxic effects of acrylic acid, methacrylic acid, their corresponding saturated carboxylic acids, HEMA, and hydroquinone on fibroblasts derived from human pulp, Dental Materials Journal 31(2) (2012) 219-225.
[52] J. Ferracane, Elution of leachable components from composites, Journal of Oral Rehabilitation 21(4) (1994) 441-452.
[53] G. Spagnuolo, V. D'Antò, C. Cosentino, G. Schmalz, H. Schweikl, S. Rengo, Effect of N-acetyl-l-cysteine on ROS production and cell death caused by HEMA in human primary gingival fibroblasts, Biomaterials 27(9) (2006) 1803-1809.
[54] K. Małkiewicz, J. Turło, A. Marciniuk-Kluska, K. Grzech-Leśniak, M. Gąsior, M. Kluska, Release of bisphenol A and its derivatives from orthodontic adhesive systems available on the European market as a potential health risk factor, Annals of Agricultural and Environmental Medicine 22(1) (2015) 172-177.
[55] N. J. Lin, C. Keeler, A. M. Kraigsley, J. Ye, S. Lin-Gibson, Effect of dental monomers and initiators on Streptococcus mutans oral biofilms, Dental Materials 34(5) (2018) 776-785.
[56] L. Sadeghinejad, D. G. Cvitkovitch, W. L. Siqueira, J. Merritt, J. P. Santerre, Y. Finer, Mechanistic, genomic and proteomic study on the effects of BisGMA-derived biodegradation product on cariogenic bacteria, Dental Materials 33(2) (2017) 175-190.
[57] C. Hansel, G. Leyhausen, U. E. H. Mai, W. Geurtsen, Effects of Various Resin Composite (Co)monomers and Extracts on Two Caries-associated Micro-organisms in vitro, Journal of Dental Research 77(1) (1998) 60-67.
[58] K. Kawai, Y. Tsuchitani, Effects of resin composite components on glucosyltransferase of cariogenic bacterium, Journal of Biomedical Materials Research 51(1) (2000) 123-127.
[59] P. Khalichi, J. Singh, D. G. Cvitkovitch, J. P. Santerre, The influence of triethylene glycol derived from dental composite resins on the regulation of Streptococcus mutans gene expression, Biomaterials 30(4) (2009) 452-459.
[60] V. Bhagat, M. L. Becker, Degradable Adhesives for Surgery and Tissue Engineering, Biomacromolecules 18(10) (2017) 3009-3039.

Additional non-limiting exemplary embodiments for the subject matter disclosed herein are provided below.

Embodiment 1 herein provides compounds of Formula (I):

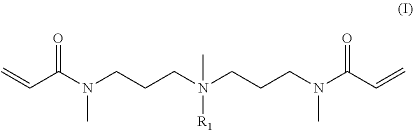

wherein R₁ is selected from the group of C₁-C₂₀ alkyl, —CH₂—CH₂—CH₂—NH—C(=O)—C=C, and —CH₂—CH₂—CH₂—N(CH₃)—C(=O)—C=C.

Embodiment 2 provides compounds of Formula (I), wherein R₁ is selected from the group of C₁-C₁₀ alkyl, —CH₂—CH₂—CH₂NH—C(=O)—C=C and —CH₂—CH₂—CH₂—N(CH₃)—C(=O)—C=C.

Embodiment 3 provides compounds of Formula (I), wherein Ri is selected from the group of C₁₀-C₂₀ alkyl.

Embodiment 4 provides compounds of Formula (I), wherein R₁ is selected from the group of C₁₀-C₁₆ alkyl.

Embodiment 5 provides compounds of Formula (I), wherein R₁ is selected from the group of C₁₂-C₁₆ alkyl.

Embodiment 6 provides the compound:

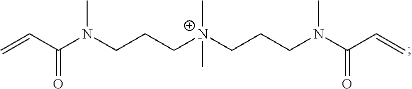 [SMILES: C=CC(=O)N(C)CCC[N+](C)(C)CCCN(C)C(=O)C=C]

or a salt thereof.

Embodiment 7 provides the compound:

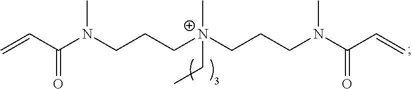

or a salt thereof.

Embodiment 8 provides the compound:

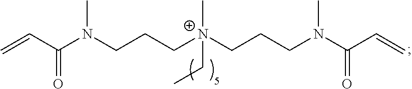

or a salt thereof.

Embodiment 9 provides the compound:

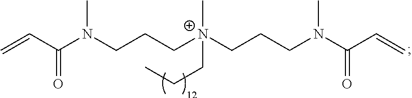

or a salt thereof.

Embodiment 10 provides a composition comprising a compound of Formula (I):

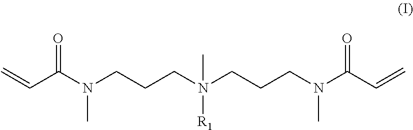

wherein R₁ is selected from the group of C₁-C₂₀ alkyl and —CH₂—CH₂—CH₂—NH—C(=O)—C=C.

Embodiment 11 provides a composition comprising a compound of Formula (I), wherein R₁ is selected from the group of C₁-C₁₀ alkyl ,—CH₂—CH₂—CH₂NH—C(=O)—C=C and —CH₂—CH₂—CH₂—N(CH₃)—C(=O)—C=C.

Embodiment 12 provides a composition comprising a compound of Formula (I), wherein R₁ is selected from the group of C₁₀-C₂₀ alkyl.

Embodiment 13 provides a composition comprising a compound of Formula (I), wherein R₁ is selected from the group of C₁₀-C₁₆ alkyl.

Embodiment 14 provides a composition comprising a compound of Formula (I), wherein R₁ is selected from the group of C₁₂-C₁₆ alkyl.

Embodiment 15 provides a composition comprising the compound:

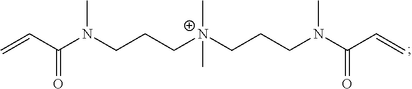 [SMILES: C=CC(=O)N(C)CCC[N+](C)(C)CCCN(C)C(=O)C=C]

or a salt thereof.

Embodiment 16 provides a composition comprising the compound:

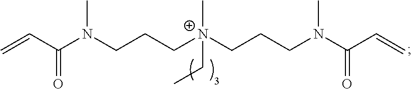

or a salt thereof.

Embodiment 17 provides a composition comprising the compound:

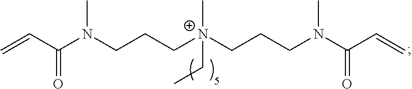

or a salt thereof.

Embodiment 18 provides a composition comprising the compound:

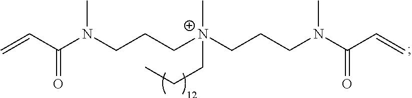

or a salt thereof.

Embodiment 19 provides a composition of any of Embodiments 10 through 18, further comprising one or more co-monomers selected from the group of bisphenol A diglycidyl ether dimethacrylate (BisGMA), triethylene glycol dimethacrylate (TEGDMA), urethane dimethacrylate (UDMA), ethylene glycol dimethylacrylate (EGDMA), ethane-1,2-diyl bis(2-methylacrylate) (PEGDMA), ethoxyiated bisphenol A dimethacrylate (EBPADMA), ethylene glycoldi(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, 1,6-hexanediol dimethacrylate (HEDMA), 1,6-hexarriethylene glycol dimethacrylate (HGDMA), divinyl benzene and derivatives thereof.

Embodiment 20 comprises the composition of Embodiment 19, wherein the co-monomer compound is BisGMA.

Embodiment 21 comprises the dental adhesive composition of Embodiment 19, wherein the co-monomer compound is TEGDMA.

Embodiment 22 comprises the dental adhesive composition of Embodiment 19, wherein the co-monomer compound is UDMA.

Embodiment 23 comprises the dental adhesive composition of Embodiment 19, wherein the co-monomer compound is EGDMA.

Embodiment 24 comprises the dental adhesive composition of Embodiment 19, wherein the co-monomer compound is PEGDMA.

Embodiment 25 comprises the composition of any of Embodiments 19 through 24, wherein the co-monomer or co-monomers comprises from about 55% to 65% of the composition, by weight.

Embodiment 26 comprises the composition of any of Embodiments 10 through 24, further comprising a polymerization initiator selected from the group of camphorquinone (CQ); trimethylbenzoyl-diphenyl-phosphine oxide (TPO); Ethyl-4-dimethylamino benzoate (EDMAB); 2,2-Dimethoxy-2-phenylacetophenone (DMPA); Bisacylphosphine oxide (BAPO); 1-Phenyl-1,2-propanedione (PPD); phosphine oxide compounds, including naphthacene (APO), 9-anthracene (APO), and bisacylphosphine oxide (BAPO); 1-phenyl-1,2-propanedione (PPD); thioxanthone (TX) and its derivatives; a dibenzoyl germanium: derivative, benzoyltrimethylgermane (BTG), dibenzoyldiethylgermane; hexaarylbiimidazole derivatives; a silane based derivative; (diethylgermanediyl)bis((4-methoxyphenyl)methanone); benzenesulfinic acid sodium salt (BS); a diaryliodonium salt, diphenyliodonium chloride or iodonium salt (diphenyliodonium hexafluorophosphate (DPIHP or DPI-PF6))], bromide, iodide, or hexafluorophosphate; benzoyl peroxide (BPO), and ethyl 4-N,N-dimethaminobenzoate.

Embodiment 27 comprises the composition of Embodiment 26, wherein the polymerization initiator is a combination of initiators selected from the group of camphorquinone/ethyl-4-(dimethylamino)benzoate (EDMAB), camphorquinone/2-(dimethylamino)ethyl methacrylate (DMAEMA)), DMPA/DPI-PF6, CQ/PPD, CQ/DMAEMA, CQ/EDMAB, CQ/DMAEMA/PDIHP, and CQ/EDMAB/DPIHP.

Embodiment 28 comprises the composition of any of Embodiments 26 through 27, wherein the polymerization initiator is one or both selected from the group of DMPA and DPI-PF.

Embodiment 29 comprises the composition of any of Embodiments 10 through 28, wherein the polymerization initiator comprises from about 0.05% to about 0.6% of the composition, by weight.

Embodiment 30 comprises the composition of any of Embodiments 10 through 29, further comprising a chemical inhibitor.

Embodiment 31 comprises the composition of Embodiment 30, wherein the chemical inhibitor is selected from the group of butylated hydroxytoluene (BHT), hydroquinone, 2,5-di-tert-butyl hydroquinone, monomethyl ether hydroquinone (MEHQ), and 2,5-di-tertiary butyl-4-methylphenol, 3,5-di-tert-butyl-4-hydroxyanisole (2,6-di-tert-butyl-4-ethoxyphenol), 2,6-di-tert-butyl-4-(dimethylamino)methylphenol or 2-(2′-hydroxy-5′-methylphenyl)-2H-benzotriazole, 2-(2′-hydroxy-5′-t -octylphenyl)-2H-benzotriazole, 2-(2′-hydroxy-4′,6′-di-tert-pentylphenyl)-2H-benzotriazole, 2-hydroxy-4-n-octoxybenzophenone, 2-(2′-hydroxy-5′-methacryloxy-ethylphenyl)-2H-benzotriazole, phenothiazine, and HALS (hindered amine light stabilizers).

Embodiment 32 comprises the composition of any of Embodiments 10 through 31, further comprising an ultraviolet light (UV) absorber.

Embodiment 33 comprises the composition of Embodiment 32, wherein the ultraviolet light (UV) absorber is selected from the group of 2-hydroxy-4-methoxybenzophenone (UV-9), 2-(2-Hydroxy-5-octylphenyl)-benzotriazole (UV-5411), salicylic acid phenyl ester, 3-(2′-hydroxy-5′-methylphenyl)benzotriazole, and 2-(2′-hydroxy-5′-methylphenyl)-benzotriazole.

Embodiment 34 comprises the composition of any of Embodiments 32 and 33, wherein the UV absorber is present in the composition at from about 0.001% to about 0,5%, by weight.

Embodiment 35 comprises the composition of any of Embodiments 32, 33, and 34, wherein the UV absorber is present in the composition at from about

Embodiment 36 comprises the composition of any of Embodiments 32, 33, and 34, wherein the UV absorber is present in the composition at from about 0.01% to about 0.5%, by weight.

Embodiment 37 comprises the composition of any of Embodiments 32 through 36, wherein the UV absorber is present in the composition at from about 0.05% to about 0.3%, by weight.

Embodiment 38 comprises the composition of any of Embodiments 32 through 37, wherein the UV absorber is present in the composition at from about 0.05% to about 0.2%, by weight.

Embodiment 39 comprises the composition of any of Embodiments 32 through 38, wherein the UV absorber is present in the composition at from about 0.05% to about 0.15%, by weight.

Embodiment 40 comprises a composition of any of Embodiments 10 through 39, further comprising one or more agents selected from the group of a fluorescent agent, a fluoride releasing agent, a radiopaque agent, a flavoring agent, and an antimicrobial agent.

Embodiment 41 comprises the composition of any of Embodiments 10 through 40, wherein the dental adhesive composition further comprises a self-etching agent.

Embodiment 42 comprises the composition of Embodiment 41, wherein the self-etching agent comprises a carboxylic acid, phosphonic acid, or phosphate groups

Embodiment 43 comprises the composition of any of Embodiments 40 and 41, wherein the self-etching agent is selected from the group of 10-methacryloyloxydecyl dihydrogen phosphate (10-MDP or MDP, CAS Reg. No. 85590-007), methacryloxyethyl hydrogen phenyl phosphate (Phenyl-P), methacryloyloxydodecylpyridinium bromide (MDPB), 4-methacryloyloxyethyl trimellitate anhydride (4-META), 4-methacryloyloxyethyl trimellitic acid (4-MET), 11-methacryloyloxy-1,1-undecanedicarboxylic acid (MAC10), 4-acryloyloxyethyl trimellitate anhydride (4-AETA), 2-methacryloyloxyethyl dihydrogen phosphate (MEP), dipentaerithritol pentaacryiate phosphate (PENTA-P), hydroxyethylmethacrylate phosphate (HEMA-P), hydroxyethylacrylate phosphate (HEA-P), bis(HEMA)-P {bis(hydroxyethylmethacrylate) phosphate), bis(HEA)-P {bis(hydroxyethylacrylate) phosphate), bis(meth)acryloxypropyl)phosphatephosphate methacrylates, acrylic ether phosphonic acid and other phosphoric acid esters.

Embodiment 44 comprises the composition of any of Embodiments 41 through 43, wherein the self-etching agent is 10-methacryloyloxydecyl dihydrogen phosphate.

Embodiment 45 comprise the composition of any of Embodiments 10 through 44, wherein the composition is a dental adhesive composition.

Embodiment 46 provides a kit, the kit comprising a useful amount of one or more of the compositions selected from any of Embodiments 1 through 45, and directions for the use of the composition.

Embodiment 47 comprises the kit of Embodiment 46, wherein a first composition comprising one or more compounds selected from any of Embodiments 1 through 18 is maintained in first container, separated from one or more co-monomers selected from any of Embodiments 19 through 25 is maintained in a second container.

What is claimed:

1. A compounds of Formula (I): 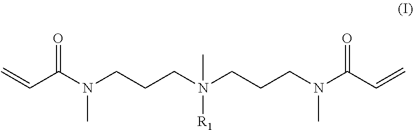
wherein R₁ is selected from the group of C₁-C₂₀ alkyl, —CH₂—CH₂—CH₂—NH—C(=O)—C=C and —CH₂—CH₂—CH₂—N(CH₃)—C(=O)—C=C.

2. The compound of claim 1, wherein R₁ is selected from the group of C₁₀-C₂₀ alkyl.

3. The compound of claim 1, wherein R₁ is selected from the group of C₁₀-C₁₆ alkyl.

4. The compound of claim 1, wherein R₁ is selected from the group of C₁₂-C₁₆ alkyl.

5. The compound of claim 1, selected from the group of: 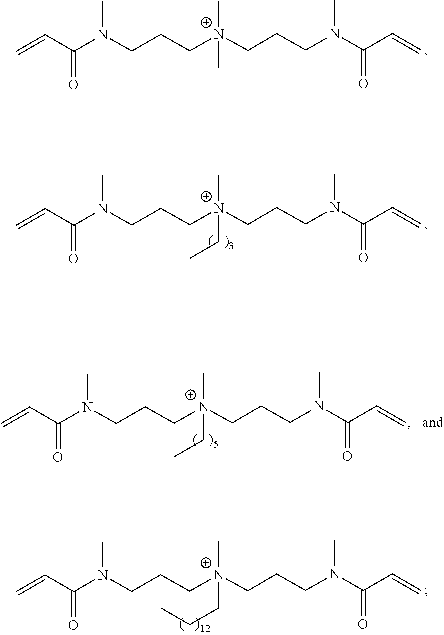
or a salt thereof.

6. A composition comprising:
a) a useful amount of a monomer compound of Formula (I), or a salt thereof: 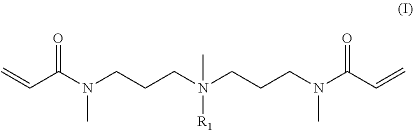
wherein R₁ is selected from the group of C₁-C₂₀ alkyl, C₁-C₂₀ alkyl, —CH₂—CH₂—CH₂—NH—C(=O)—C=C and —CH₂—CH₂—CH₂—N(CH₃)—C(=O)—C=C; and
b) a useful amount of one or more co-monomers selected from the group of bisphenol A diglycidyl ether dimethacrylate (BisGMA), triethylene glycol dimethacrylate (TEGDMA), urethane dimethacrylate (UDMA), ethylene glycol dimethylacrylate (EGDMA), ethane-1,2-diyl bis(2-methylacrylate) (PEGDMA), ethoxylated bisphenol A dimethacrylate (EBPADMA), ethylene glycoldi(meth)acrylate, hexanediol di(meth)acrylate, tripropylene glycol di(meth)acrylate, butanediol di(meth)acrylate, neopentyl glycol di(meth)acrylate, diethylene glycol di(meth)acrylate, triethylene glycol di(meth)acrylate, dipropylene glycol di(meth)acrylate, allyl (meth)acrylate, 1,6-hexanediol dimethacrylate (HEDMA), 1,6-hexamethylene glycol dimethacrylate (HGDMA), and divinyl benzene.

7. The composition of claim 6, wherein the monomer of Formula (1), or a salt thereof, comprises from about 35% to about 45%, by weight, of the composition and the co-monomer or co-monomers comprise from about 55% to 65% of the composition.

8. The composition of claim 7, wherein the co-monomer is one or more agents selected from the group of BisGMA, TEGDMA, UDMA, EGDMA, PEGDMA.

9. composition of claim 8, wherein the compound of Formula (I) is selected from the group of: 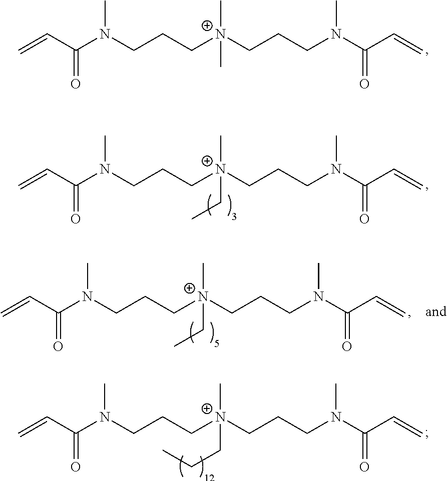
or a salt thereof.

10. The composition of claim 8, further comprising polymerization initiator selected from the group of camphorquinone (CQ); trimethylbenzoyl-diphenyl-phosphine oxide (TPO); Ethyl-4-dimethylamino benzoate (EDMAB); 2,2-Dimethoxy-2-phenylacetophenone (DMPA); Bisacylphosphine oxide (BAPO); 1-anthracene
Phenyl-1,2-propanedione (PPD); phosphine oxide compounds, including naphthacene (APO), 9-(APO), and bisacylphosphine oxide (BAPO); 1-phenyl-1,2-propanedione (PPD); thioxanthone (TX) and its derivatives; a dibenzoyl germanium derivative, benzoyltrimethylgermane (BTG), dibenzoyldiethylgermane; hexaarylbiimidazole derivatives; (diethylgermanediyl)bis((4-methoxyphenyl)methanone); benzenesulfinic acid sodium salt (BS); a diaryliodonium salt, diphenyliodonium chloride or iodonium salt [diphenyliodonium hexafluorophosphate (DPIHP or DPI-PF6))], bromide, iodide, or hexafluorophosphate; benzoyl peroxide (BPO), and ethyl 4-N,N-dimethaminobenzoate.

11. The composition of claim 12, wherein the polymerization initiator is one or more agents selected from the group of DMPA and DPI-PF6.
